SEQUENCE LISTING (14 sequences)

SEQ ID NO: 1 - protein, 5 aa, Artificial Sequence
  Synthetic peptide
SRGDS

SEQ ID NO: 2 - protein, 4 aa, Artificial Sequence
  Synthetic peptide
RGDS

SEQ ID NO: 3 - protein, 13 aa, Artificial Sequence
  Synthetic peptide
AGQWHRVSVRWGC

SEQ ID NO: 4 - protein, 7 aa, Artificial Sequence
  Synthetic peptide
SVVYGLR

SEQ ID NO: 5 - protein, 4 aa, Artificial Sequence
  Synthetic peptide
DGEA

SEQ ID NO: 6 - protein, 8 aa, Artificial Sequence
  Synthetic peptide
IRIKIRIK

SEQ ID NO: 7 - protein, 12 aa, Artificial Sequence
  Synthetic peptide
IKKIIKKIIKKI

SEQ ID NO: 8 - protein, 9 aa, Artificial Sequence
  Synthetic peptide
  MOD_RES (3)..(3): Hyp
  MOD_RES (6)..(6): Hyp
  MOD_RES (9)..(9): Hyp
GPXGPXGPX

SEQ ID NO: 9 - protein, 9 aa, Artificial Sequence
  Synthetic peptide
  MOD_RES (2)..(2): Hyp
  MOD_RES (5)..(5): Hyp
  MOD_RES (8)..(8): Hyp
PXGPXGPXG

SEQ ID NO: 10 - protein, 4 aa, Artificial Sequence
  Synthetic peptide
IRIK

SEQ ID NO: 11 - protein, 9 aa, Artificial Sequence
  Synthetic peptide
  MOD_RES (1)..(1): Hyp
  MOD_RES (4)..(4): Hyp
  MOD_RES (7)..(7): Hyp
XPGXPGXPG

SEQ ID NO: 12 - protein, 9 aa, Artificial Sequence
  Synthetic peptide
  MOD_RES (2)..(2): Hyp
  MOD_RES (5)..(5): Hyp
  MOD_RES (8)..(8): Hyp
GXPGXPGXP

SEQ ID NO: 13 - protein, 9 aa, Artificial Sequence
  Synthetic peptide
  MOD_RES (1)..(1): Hyp
  MOD_RES (4)..(4): Hyp
  MOD_RES (7)..(7): Hyp
XGPXGPXGP

SEQ ID NO: 14 - protein, 9 aa, Artificial Sequence
  Synthetic peptide
  MOD_RES (3)..(3): Hyp
  MOD_RES (6)..(6): Hyp
  MOD_RES (9)..(9): Hyp
PGXPGXPGX

(19) United States
(12) Patent Application Publication
(10) Pub. No.: US 2023/0416468 A1
(43) Pub. Date: Dec. 28, 2023

(54) BIOACTIVE POLYETHYLENE COPOLYMER, POLYETHYLENE MACROMOLECULE AND RELATED METHODS THEREOF

(71) Applicants: Agency for Science, Technology and Research, Singapore (SG); Mitsui Chemicals, Inc., Tokyo (JP)

(72) Inventors: Peili Teo, Singapore (SG); Haruyuki Makio, Tokyo (JP); Jiayi Guo, Singapore (SG); Eun Ju Park, Singapore (SG); Yew Chin Teo, Singapore (SG)

(21) Appl. No.: 18/251,108

(22) PCT Filed: Oct. 30, 2020
(86) PCT No.: PCT/SG2020/050622

§ 371 Date: Apr. 28, 2023

(51) Int. Cl. C08G 81/02; A61K 31/787

(52) U.S. Cl. CPC C08G 81/024; A61K 31/787

(57) ABSTRACT

There is provided a bioactive polyethylene copolymer with a poly(norbornene) backbone comprising one or more repeating units represented by general formula (I) and one or more repeating units represented by general formula (II). Also provided are a polyethylene macromolecule, a material comprising said bioactive polyethylene copolymer, a method of preparing said bioactive polyethylene copolymer and a method of preparing said polyethylene macromolecule.

### TECHNICAL FIELD

The present disclosure relates broadly to a bioactive polyethylene copolymer, a polyethylene macromolecule and a material comprising said bioactive polyethylene copolymer. The present disclosure also relates to methods of preparing said bioactive polyethylene copolymer, said polyethylene macromolecule and said material.

### BACKGROUND

A better understanding of the biology and physiology of living things over the years has led to an appreciation of the potential of using alternative materials to enhance or replace existing functions in biological systems.

However, identifying a suitable material that meets both the mechanical and biological requirements to function desirably in or with biological systems is often challenging.

This is because bioactive molecules (e.g. collagen, chitosan etc) that have the desired biological attributes often lack the mechanical strength needed for a useful biomedical application. For example, many of such bioactive molecules are very hygroscopic and exist as gels upon absorption of moisture, rendering them too weak for use in weight bearing biomedical applications such as implantable devices on their own.

On the other hand, synthetic materials that have mechanically superior characteristics lack the biological attributes needed for them to be properly used in applications that require constant interaction with biological systems.

Of particular interest is polyethylene (PE) that is often used in biomedical applications despite its modest biocompatibility. PE is a common plastic frequently used in consumer care and disposable products (e.g., diapers and sanitary products), and medical devices (e.g., biliary stents, gastrointestinal stents, arthroplasty implants and joint implants, in the form of either high density polyethylene (HDPE) or cross-linked polyethylene (XPE)) due to properties such as inertness, high mechanical strength, good thermal stability, good/easy material processability, ease of sterilization and low cost.

However, there are several critical issues associated with the use of polyethylene. For example, polyethylene implants can induce foreign body reaction (FBR) when inserted into a human body. This can result in inflammation and other types of undesired immune responses being elicited around the implantation site, which would require immunosuppressant administration to bring down the inflammation. In the event that immunosuppressant administration fails, implant removal may be necessary together with continued immunosuppressant administration. In polyethylene-based gastrointestinal stents and biliary stents, biofouling can also be a problem which results in stent occlusion and the need for re-stenting. Skin sensitization is also common with polyethylene-based products as a result of friction against the material. For example, diaper dermatitis is the most common skin condition experienced by infants which can lead to ulceration and pustule formation in severe cases. In adults who require diaper wearing for reasons such as incontinence and reduced mobility, dermatitis may also result in pressure ulcers which often lead to sepsis. Further, working with polyethylene is also challenging as the synthetic polymer is not only insoluble in most organic solvents, it is also a relatively inert polymer on its own.

Combining these different materials with the hope that the resultant material obtained can achieve both the desired biological and mechanical properties is also challenging. This is because bioactive molecules such as peptides and carbohydrates are often incompatible with polyethylene since the former is hydrophilic whereas the latter is hydrophobic.

Thus, physically blending the two different materials together often result in phase separation of the two mutually incompatible materials, rendering the obtained entire material ineffective.

The inherent differences in their hydrophilicity likewise make chemically synthesising a bioactive polyethylene copolymer from these materials extremely difficult, especially when the molecular weights of these materials are relatively high. This is in addition to the various complex chemical hurdles (e.g. low reactivity due to inertness of polyethylene, unwanted chemical leaching of by-products etc) that need to be overcome when attempting to chemically combine these two chemically different types of materials together.

In view of the above, there is a need to address or at least ameliorate the above-mentioned problems. In particular, there is a need to provide a bioactive polyethylene copolymer, a polyethylene macromolecule, a material comprising said bioactive polyethylene copolymer and related methods that address or at least ameliorate the above-mentioned problems.

### SUMMARY

In one aspect, there is provided a bioactive polyethylene copolymer with a poly(norbornene) backbone comprising one or more repeating units represented by general formula (I) and one or more repeating units represented by general formula (II):

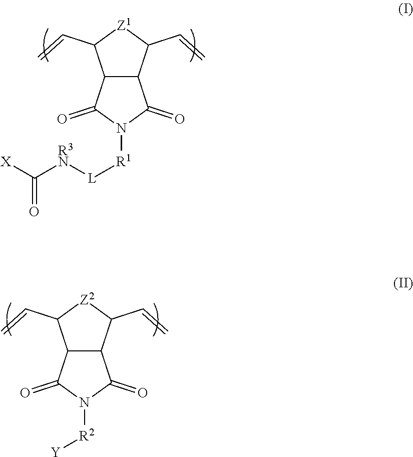

wherein

R¹ is optionally substituted alkyl;

R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;

R³ is selected from H, optionally substituted alkyl, optionally substituted alkenyl or optionally substituted alkynyl;

L is heteroalkylene;

X comprises a bioactive moiety selected from the group consisting of proteins, peptides, carbohydrates, therapeutic/drug molecules and derivatives thereof;

Y comprises polyethylene or parts thereof; and

Z¹ and Z² are each independently selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl.

In one embodiment, Y is represented by general formula (III):

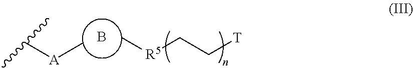

wherein

A is optionally present as NR^{c}, wherein R^{c} is independently selected from H, optionally substituted alkyl, optionally substituted alkenyl or optionally substituted alkynyl;

B is optionally present as a 5-membered or 6-membered heterocyclic ring having at least one N heteroatom in the ring;

R⁵ is selected from an optionally substituted alkyl, optionally substituted alkenyl, optionally substituted alkynyl, optionally substituted alkoxyalkyl, optionally substituted alkylcarbonyl or optionally substituted alkylcarbonylalkyl;

T is a terminal group selected from the group consisting of hydrogen and methyl; and

n is from 10 to 350.

In one embodiment, n is from 20 to 250.

In one embodiment, B is present and represented by the following structure:

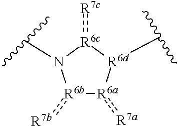

wherein

R^{6a}, R^{6b}, R^{6c} and R^{6d} are each independently selected from the group consisting of C, CR^{a}, CR^{a}R^{b}, N, NR^{c}, O or S, wherein R^{a}, R^{b}, and R^{c} are each independently selected from the group consisting of H, optionally substituted alkyl, optionally substituted alkenyl and optionally substituted alkynyl; and R^{7a}, R^{7b} and R^{7c} are optionally present as ═O, ═S, —F, —Cl, —Br, —I. ═CR^{a}R^{b}, —CR^{a}R^{b}R^{c}, —OH, —SH, —NH₂ or ═NR^{c}.

In one embodiment, Y is selected from the following general formulae (IIIa), (IIIb) or (IIIc):

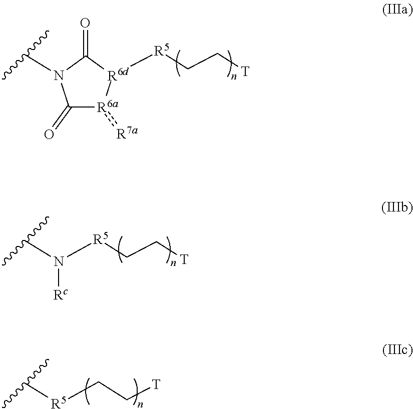

wherein

R⁵ is selected from the group consisting of C₁-C₂₀ alkyl, C₂-C₂₀ alkenyl, C₂-C₂₀ alkynyl, C₁-C₂₀ alkoxy, C₁-C₂₀ alkoxyalkyl, C₂-C₂₀ alkylcarbonyl and C₃-C₂₀ alkylcarbonylalkyl;

R^{6a} and R^{6d} are each independently selected from the group consisting of C, CR^{a}, CR^{a}R^{b}, N, NR^{c}, O or S, wherein R^{a}, R^{b}, and R^{c} are each independently selected from the group consisting of H, optionally substituted alkyl, optionally substituted alkenyl and optionally substituted alkynyl;

R^{7a} is optionally present as ═O, ═S, —F, —Cl, —Br, —I, ═CR^{a}R^{b}, —CR^{a}R^{b}R^{c}, —OH, —SH, —NH₂ or ═NR^{c};

T is a terminal group selected from the group consisting of hydrogen and methyl; and

n is from 10 to 350.

In one embodiment, Y is selected from the following general formulae (IIId), (IIIe) or (IIIf):

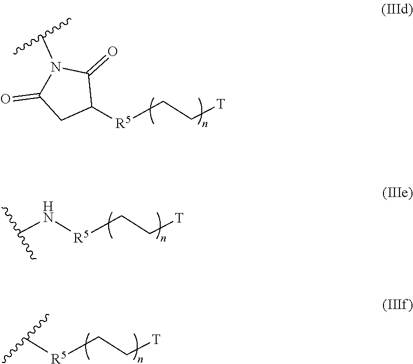

wherein

R⁵ is selected from the group consisting of C₁-C₂₀ alkyl, C₂-C₂₀ alkenyl, C₂-C₂₀ alkynyl, C₁-C₂₀ alkoxy, C₁-C₂₀ alkoxyalkyl, C₂-C₂₀ alkylcarbonyl and C₃-C₂₀ alkylcarbonylalkyl;

T is a terminal group selected from the group consisting of hydrogen and methyl; and

n is from 10 to 350.

In one embodiment, the repeating unit represented by general formula (I) is in an amount of from 1 to 100 molar % relative to the copolymer.

In one embodiment, the molecular weight of general formula (I) do not differ from the molecular weight of general formula (II) by more than 30% of the molecular weight of general formula (II).

In one embodiment, L is heteroalkylene having from 20 carbon atoms to 300 carbon atoms.

In one embodiment, L is polyethylene glycol (PEG).

In one embodiment, L is polyethylene glycol (PEG) having a number average molecular weight of between 500 and 7,000.

In one embodiment, R¹ is C₁-C₄ alkyl and R² is selected from C₁-C₂₀ alkyl, C₂-C₂₀ alkenyl, C₂-C₂₀ alkynyl, C₁-C₂₀ alkoxy, C₁-C₂₀ alkoxyalkyl, C₂-C₂₀ alkylcarbonyl or C₃-C₂₀ alkylcarbonylalkyl.

In one embodiment, R¹ is straight or branched C₁-C₄ alkyl substituents independently selected from methyl, ethyl, n-propyl, 2-propyl, isopropyl, n-butyl, isobutyl, sec-butyl or t-butyl, and R² is straight or branched C₁-C₂₀ alkyl substituents independently selected from methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl or decyl.

In one embodiment, Z¹ and Z² are both CR^{a}R^{b} wherein R^{a} and R^{b} are each independently selected from the group consisting of H, optionally substituted alkyl, optionally substituted alkenyl and optionally substituted alkynyl.

In one embodiment, X comprises protein, peptide or carbohydrate selected from the group consisting of peptide sequence, laminin-derived peptide, integrin binding peptide, cell-penetrating peptide, collagen sequence, collagen mimics, collagen fragment, heparin sulfate, glycosaminoglycans (GAGs) and derivatives thereof.

In one embodiment, X is selected from the group consisting of RGD, SRGDS, RGDS, A5G81 (AGQWHRVSVRWGC), SVVYGLR, (IRIK)₂, (IKKI)₃, DGEA, (PHypG)ₙ type sequence, (PGHyp)ₙ type sequence, (HypGP)ₙ type sequence, (HypPG)ₙ type sequence, (GHypP)ₙ type sequence, (GPHyp)ₙ type sequence, heparin oligosaccharide DP8, DP10, DP12, DP14, DP16 and hyaluronic acid.

In one aspect, there is provided a method of preparing a bioactive polyethylene copolymer disclosed herein, the method comprising: polymerising one or more bioactive macromolecules represented by general formula (IV) with one or more polyethylene macromolecules represented by general formula (V) in the presence of a catalyst to obtain the bioactive polyethylene copolymer:

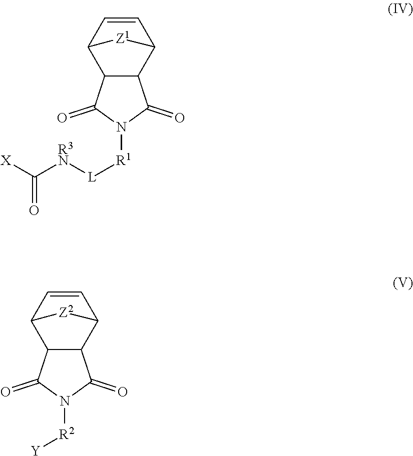

wherein

R¹ is optionally substituted alkyl;

R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;

R³ is selected from H, optionally substituted alkyl, optionally substituted alkenyl or optionally substituted alkynyl;

L is heteroalkylene;

X comprises a bioactive moiety selected from the group consisting of proteins, peptides, carbohydrates, therapeutic/drug molecules and derivatives thereof;

Y comprises polyethylene or parts thereof; and

Z¹ and Z² are each independently selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl.

In one embodiment, the catalyst comprises a ruthenium complex.

In one embodiment, the method comprises ring opening metathesis polymerisation (ROMP).

In one aspect, there is provided a polyethylene macromolecule represented by general formula (VIII) for preparing the copolymer disclosed herein:

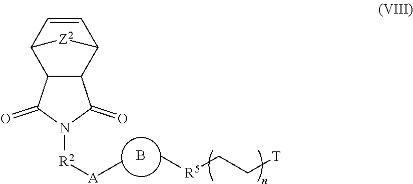

wherein

R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;

Z² is selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl;

A is optionally present as NR^{c}, wherein R^{c} is independently selected from H, optionally substituted alkyl, optionally substituted alkenyl or optionally substituted alkynyl;

B is optionally present as a 5-membered or 6-membered heterocyclic ring having at least one N heteroatom in the ring;

R⁵ is selected from an optionally substituted alkyl, optionally substituted alkenyl, optionally substituted alkynyl, optionally substituted alkoxyalkyl, optionally substituted alkylcarbonyl or optionally substituted alkylcarbonylalkyl;

T is a terminal group selected from the group consisting of hydrogen and methyl; and

n is from 10 to 350.

In one aspect, there is provided a method of preparing a polyethylene macromolecule disclosed herein, the method comprising:
(i) providing a dicarboxylic anhydride having general formula (IX):

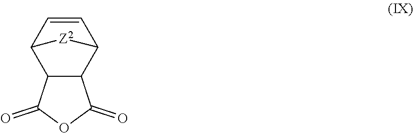

wherein Z² is selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl; and
(ii) reacting said dicarboxylic anhydride having general formula (IX) with an amine to obtain the polyethylene macromolecule, the amine is represented by general formula (X):

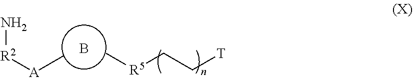

wherein

R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;

A is optionally present as NR^{c}, wherein R^{c} is independently selected from H, optionally substituted alkyl, optionally substituted alkenyl or optionally substituted alkynyl;

B is optionally present as a 5-membered or 6-membered heterocyclic ring having at least one N heteroatom in the ring;

R⁵ is selected from the group consisting of C₁-C₂₀ alkyl, C₂-C₂₀ alkenyl, C₂-C₂₀ alkynyl, C₁-C₂₀ alkoxy, C₁-C₂₀ alkoxyalkyl, C₂-C₂₀ alkylcarbonyl and C₃-C₂₀ alkylcarbonylalkyl;

T is a terminal group selected from the group consisting of hydrogen and methyl;

n is from 10 to 350.

In one embodiment, the method further comprising, prior to step (ii), (a-i) providing a polyethylene having general formula (XIa) or (XIb):

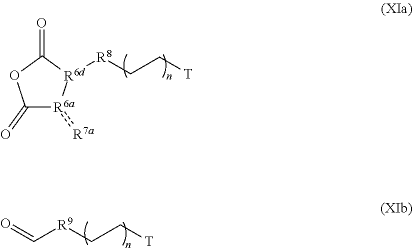

wherein

R^{6a} and R^{6d} are each independently selected from the group consisting of C, CR^{a}, CR^{a}R^{b}, N, NR^{c}, O or S, wherein R^{a}, R^{b}, and R^{c} are each independently selected from the group consisting of H, optionally substituted alkyl, optionally substituted alkenyl and optionally substituted alkynyl;

R^{7a} is optionally present as ═O, ═S, —F, —Cl, —Br, —I, ═CR^{a}R^{b}, —CR^{a}R^{b}R^{c}, —OH, —SH, —NH₂ or ═NR^{c};

R⁸ and R⁹ are each independently selected from the group consisting of C₁-C₂₀ alkyl, C₂-C₂₀ alkenyl, C₂-C₂₀ alkynyl, C₁-C₂₀ alkoxy, C₁-C₂₀ alkoxyalkyl, C₂-C₂₀ alkylcarbonyl and C₃-C₂₀ alkylcarbonylalkyl;

T is a terminal group selected from the group consisting of hydrogen and methyl;

n is from 10 to 350; and
(b-i) reacting said polyethylene having general formula (XIa) or (XIb) with a diamine H₂N—R²—NH₂ or ammonia NH₃ to obtain the amine having general formula (X).

In one embodiment, at least one of step (ii) and step (b-i) is performed in the presence of an organic solvent and/or a base.

In one embodiment, the organic solvent comprises an aromatic solvent; and the base comprises a tertiary amine.

In one aspect, there is provided a material comprising a copolymer disclosed herein for use in medicine.

In one embodiment, the material is part of an apparatus selected from the group consisting of consumer care products, wound dressing, skin scaffold, bone and bone marrow organoid scaffold, cartilage implant, joint implant and medical device.

### Definitions

The term “polymer” as used herein refers to a chemical compound comprising repeating units and is created through a process of polymerization. The units composing the polymer are typically derived from monomers and/or macromonomers. A polymer typically comprises repetition of a number of constitutional units.

The terms “monomer” or “macromonomer” as used herein refer to a chemical entity that may be covalently linked to one or more of such entities to form a polymer.

The term “bioactive” as used herein broadly refers to the property of having a biological effect, preferably a desirable or positive biological effect on a living organism, tissue, or cell.

The term “biocompatible” as used herein broadly refers to a property of being compatible with biological systems or parts of the biological systems without substantially or significantly eliciting an adverse physiological response such as a toxic reaction, an immune reaction, an injury or the like. Such biological systems or parts include blood, cells, tissues, organs or the like.

The term “bond” refers to a linkage between atoms in a compound or molecule. The bond may be a single bond, a double bond, or a triple bond.

In the definitions of a number of substituents below, it is stated that “the group may be a terminal group or a bridging group”. This is intended to signify that the use of the term is intended to encompass the situation where the group is a terminal group/moiety as well as the situation where the group is a linker between two other portions of the molecule. Using the term “alkyl” having 1 carbon atom as an example, it will be appreciated that when existing as a terminal group, the term “alkyl” having 1 carbon atom may mean —CH₃ and when existing as a bridging group, the term “alkyl” having 1 carbon atom may mean —CH₂— or the like.

The term “alkyl” as a group or part of a group refers to a straight or branched aliphatic hydrocarbon group having 1 to 20 carbon atoms, 1 to 10 carbon atoms, 1 to 6 carbon atoms, or 1, 2, 3, 4, 5, 6, 7, 8, 9, 10, 11, 12, 13, 14, 15, 16, 17, 18, 19 or 20 carbon atoms. Examples of suitable straight and branched alkyl substituents include methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl and the like. The group may be a terminal group or a bridging group.

The term “alkenyl” as a group or part of a group denotes an aliphatic hydrocarbon group containing at least one carbon-carbon double bond and which may be straight or branched having 2 to 20 carbon atoms, 2 to 10 carbon atoms, 2 to 6 carbon atoms, or 2, 3, 4, 5, 6, 7, 8, 9, 10, 11, 12, 13, 14, 15, 16, 17, 18, 19 or 20 carbon atoms in the chain. The group may contain a plurality of double bonds and the orientation about each double bond is independently E or Z. Exemplary alkenyl groups include, but are not limited to, ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 2-octenyl, 3-octenyl, 1-nonenyl, 2-nonenyl, 3-nonenyl, 1-decenyl, 2-decenyl, 3-decenyl and the like. The group may be a terminal group or a bridging group.

The term “alkynyl” as a group or part of a group denotes an aliphatic hydrocarbon group containing at least one carbon-carbon triple bond and which may be straight or branched having 2 to 20 carbon atoms, 2 to 10 carbon atoms, 2 to 6 carbon atoms, or 2, 3, 4, 5, 6, 7, 8, 9, 10, 11, 12, 13, 14, 15, 16, 17, 18, 19 or 20 carbon atoms in the chain. The group may contain a plurality of triple bonds. Exemplary alkynyl groups include, but are not limited to, acetylenyl, propynyl, 1-butynyl, 2-butynyl, 3-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl and the like. The group may be a terminal group or a bridging group.

The term “heteroalkylene” as used herein refers to alkylene having one or more —CH₂— replaced with a heteroatom selected from O, NR, Si, P or S, where R is hydrogen or alkyl as defined herein. The term “heteroalkylene” can be linear, branched or cyclic and containing up to 500 carbon atoms.

The term “alkoxy” as used herein refers to straight chain or branched alkyloxy groups. Examples include methoxy, ethoxy, n-propoxy, isopropoxy, tert-butoxy, and the like.

The term “alkoxyalkyl” as used herein is intended to broadly refer to a group containing —R—O—R′, where R and R′ are alkyl as defined herein. The group may be a terminal group or a bridging group.

The term “alkylcarbonyl” as used herein is intended to broadly refer to a group containing —R—C(═O)—, where R is alkyl as defined herein. The group may be a terminal group or a bridging group.

The term “alkylcarbonylalkyl” as used herein is intended to broadly refer to a group containing —R—C(═O)—R′, where R and R′ are alkyl as defined herein. The group may be a terminal group or a bridging group.

The term “carboxylalkyl” as used herein is intended to broadly refer to a group containing —C(═O)—O—R, where R is alkyl as defined herein. The group may be a terminal group or a bridging group.

The term “oxycarbonylalkyl” as used herein is intended to broadly refer to a group containing —O—C(═O)—R, where R is alkyl as defined herein. The group may be a terminal group or a bridging group.

The term “alkylcarboxylalkyl” as used herein is intended to broadly refer to a group containing —R—C(═O)—O—R′, where R and R′ are alkyl as defined herein. The group may be a terminal group or a bridging group.

The term “alkoxycarbonylalkyl” as used herein is intended to broadly refer to a group containing —R—O—C(═O)—R′, where R and R′ are alkyl as defined herein. The group may be a terminal group or a bridging group.

The term “oxy” as used herein is intended to broadly refer to a group containing —O—.

The term “carbonyl” as used herein is intended to broadly refer to a group containing —C(═O)—.

The term “oxycarbonyl” as used herein is intended to broadly refer to a group containing —O—C(═O)—.

The term “carboxyl” as used herein is intended to broadly refer to a group containing —C(═O)—O—R, where R is hydrogen or an organic group.

The term “halogen” represents chlorine, fluorine, bromine or iodine. The term “halo” represents chloro, fluoro, bromo or iodo.

The term “amine group” or the like is intended to broadly refer to a group containing —NR₂, where R is independently a hydrogen or an organic group. The group may be a terminal group or a bridging group.

The term “amide group” or the like is intended to broadly refer to a group containing —C(═O)NR₂, where R is independently a hydrogen or an organic group. The group may be a terminal group or a bridging group.

The term “heterocyclic” as used herein broadly refers to a structure where two or more different kinds of atoms are connected to form at least one ring. For example, a heterocyclic ring may be formed by carbon atoms and at least another atom (i.e. heteroatom) selected from oxygen (O), nitrogen (N) or (NR) and sulfur (S), where R is independently a hydrogen or an organic group. The term also includes, but is not limited to, saturated and unsaturated 5-membered, and saturated and unsaturated 6-membered rings. Examples of groups having a heterocyclic structure include, but are not limited to furan, thiophene, 1H-pyrrole, 2H-pyrrole, 1-pyrroline, 2-pyrroline, 3-pyrroline, 1-pyrazoline, 2-pyrazoline, 3-pyrazoline, 2-imidazoline, 3-imidazoline, 4-imidazoline, pyrazole, imidazole, oxazole, isoxazole, thiazole, isothiazole, 1,2,3-triazole, 1,2,4-triazole, 1,2,3-oxadiazole, disubstituted 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, tetrahydrofuran, tetrahydrothiophene, pyrrolidine, 1,3-dioxolane, 1,2-oxathiolane, 1,3-oxathiolane, pyrazolidine, imidazolidine, pyridine, pyridazine, pyrimidine, pyrazine, 1,2-oxazine, 1,3-oxazine, 1,4-oxazine, thiazine, 1,2,3-triazine, 1,2,4-triazine, 1,3,5-triazine, 2H-pyran, 4H-pyran, 2-pyrone, 4-pyrone, 1,4-dioxin, 2H-thiopyran, 4H-thiopyran, tetrahydropyran, thiane, piperidine, 1,4-dioxane, 1,2-dithiane, 1,3-dithiane, 1,4-dithiane, 1,3,5-trithiane, piperazine, morpholine, thiomorpholine and the like.

The term “optionally substituted,” when used to describe a chemical structure or moiety, refers to the chemical structure or moiety wherein one or more of its hydrogen atoms is optionally substituted with a chemical moiety or functional group such as alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-).

The term “micro” as used herein is to be interpreted broadly to include dimensions from about 1 micron to about 1000 microns.

The term “nano” as used herein is to be interpreted broadly to include dimensions less than about 1000 nm, less than about 500 nm, less than about 100 nm or less than about 50 nm.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed there between.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of a bioactive polyethylene copolymer, a polyethylene macromolecule for preparing the bioactive polyethylene copolymer, a material comprising the bioactive polyethylene copolymer and related methods are disclosed hereinafter.

### Bioactive Polyethylene Copolymer

There is provided a bioactive polyethylene copolymer with a poly(norbornene) backbone comprising one or more repeating units represented by general formula (I) and one or more repeating units represented by general formula (II):

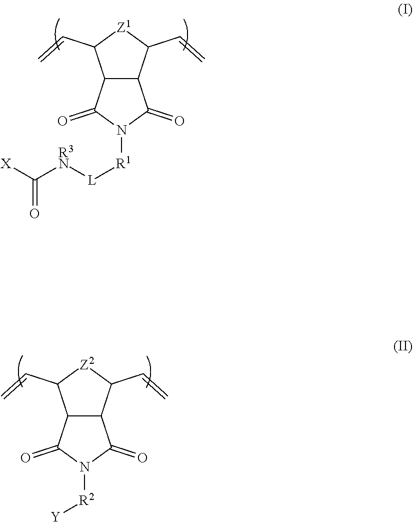

wherein

R¹ is optionally substituted alkyl;

R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;

R³ is selected from H, optionally substituted alkyl, optionally substituted alkenyl or optionally substituted alkynyl;

L is heteroalkylene;

X comprises a bioactive moiety selected from the group consisting of proteins, peptides, carbohydrates, therapeutic/drug molecules and derivatives thereof;

Y comprises polyethylene or parts thereof; and

Z¹ and Z² are each independently selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl.

In various embodiments, the repeating unit(s) represented by general formula (I) and/or moiety X possess bioactivity, biocompatibility and/or biodegradability. In various embodiments, the repeating unit(s) represented by general formula (II) and/or moiety Y possess good mechanical strength/hardness/compatibility/miscibility with base polyolefin materials on its own or when used as additives to the base materials. In various embodiments, the repeating unit represented by general formula (II) and/or moiety Y has a higher mechanical strength than the repeating unit represented by general formula (I) and/or moiety X. Advantageously, the presence of repeating units represented by general formulae (I) and (II) in the bioactive polyethylene copolymer imparts both bioactivity and mechanical strength to the copolymer, leading to a mechanically strong bioactive copolymer. In various embodiments, the copolymer may also be biocompatible. Accordingly, in various embodiments, the copolymer is capable of being classified as a biomaterial. Advantageously, due to the presence of polyethylene side chains, the bioactive polyethylene copolymer has a higher thermal stability than conventional biomolecules or biomaterials such as pure collagen. Even more advantageously, the thermal stability of the bioactive polyethylene copolymer allows for embodiments of the copolymer to be suitable for processing at high temperatures or even harsh material processing such as melt extrusion and melt blowing >200° C., making the copolymer ideal/attractive for use in applications such as biomedical devices or non-woven fiber/fabrics which is a key component of diapers. In various embodiments, the repeating unit represented by general formula (II) and/or moiety Y is substantially or completely non-bioactive, or at least less bioactive than the repeating unit represented by general formula (I) and/or bioactive moiety X.

In various embodiments, L is a polymeric linker that links the bioactive moiety X to the poly(norbornene) backbone. Advantageously, L is designed to be adjustable and/or customizable based on the size of the bioactive moiety X and the size/length of the synthetic polymer (i.e. polyethylene) present in Y. In various embodiments, the physical properties of the copolymer can be changed or tuned, depending on the length of L (e.g., PEG chain) in the macromonomer or polymers. The molecular weight and/or length of the polymeric linker L may be customized to suit the molecular weight and/or length of the bioactive moiety X and synthetic polymer (i.e. polyethylene) chosen for Y, depending on the application the copolymer is to be used for. In various embodiments, the molecular weight of Y is no more than about 5,000. For example, for applications not needing high strength such as non-woven cloth for wound dressings and diapers, PE of molecular weight under 5,000 can be used as the resultant copolymers would be blended with moderate molecular weight PE for fiber production. In other embodiments, the molecular weight of Y is more than about 5,000. For example, for applications requiring high mechanical strength such as cartilage and joint implants where the copolymer would be blended with ultra high molecular weight polyethylene (UHMWPE) for implant fabrication, molecular weight of Y is 5,000 and above.

In various embodiments, Y is represented by general formula (III):

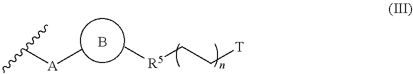

wherein

A is optionally present as NR^{c}, wherein R^{c} is independently selected from H, optionally substituted alkyl, optionally substituted alkenyl or optionally substituted alkynyl;

B is optionally present as a 5-membered or 6-membered heterocyclic ring having at least one N heteroatom in the ring;

R⁵ is selected from an optionally substituted alkyl, optionally substituted alkenyl, optionally substituted alkynyl, optionally substituted alkoxyalkyl, optionally substituted alkylcarbonyl or optionally substituted alkylcarbonylalkyl; and

T is a terminal group selected from the group consisting of hydrogen and methyl.

In various embodiments, Y is represented by general formula (III-1):

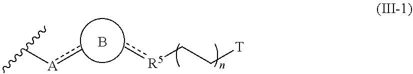

wherein

A is optionally present as N or NR^{c}, wherein R^{c} is independently selected from H, optionally substituted alkyl, optionally substituted alkenyl or optionally substituted alkynyl;

B is optionally present as a 5-membered or 6-membered heterocyclic ring having at least one N heteroatom in the ring;

R⁵ is selected from an optionally substituted alkyl, optionally substituted alkenyl, optionally substituted alkynyl, optionally substituted alkoxyalkyl, optionally substituted alkylcarbonyl or optionally substituted alkylcarbonylalkyl;

T is a terminal group selected from the group consisting of hydrogen and methyl; and wherein the dotted lines represent optional chemical bonds.

In various embodiments, the dotted line between A and B represents an optional second chemical bond. In various embodiments, the dotted line between B and R⁵ represents an optional second chemical bond.

In various embodiments, carbons of the original PE back bone are even number.

In various embodiments, n is from about 10 to about 350, from about 20 to about 250, or from about 25 to about 100. In various embodiments, PE having from about 10 to about 350 repeating units (n) is suitable for use as a starting material. In various embodiments, PE having from about 20 to about 250 repeating units (n) or from about 25 to about 100 repeating units (n) is further suitable for use/handling in macromonomer synthesis and polymerization. In these ranges of n, polyethylene (PE) has good mechanical properties and compatibility with/to base polyolefin materials, while maintaining sufficient reactivity during the synthesis and copolymerization of the macromonomers. In various embodiments, n is at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 105, at least about 110, at least about 115, at least about 120, at least about 125, at least about 130, at least about 135, at least about 140, at least about 145, at least about 150, at least about 155, at least about 160, at least about 165, at least about 170, at least about 175, at least about 180, at least about 185, at least about 190, at least about 195, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, or at least about 350. It will be appreciated by a person skilled in the art that in various embodiments, the value of n represents the average number of repeating units present in the polymer or polymer mixtures. In various embodiments, n also represents the degree of polymerization.

In various embodiments, A is present while B is absent from general formula (III-1). In such embodiments, Y is represented by general formula (III-2):

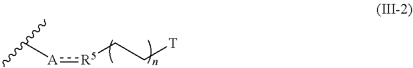

In various embodiments, A is present as N or NR^{c}, wherein R^{c} is independently selected from H, optionally substituted C₁-C₂₀ alkyl, optionally substituted C₂-C₂₀ alkenyl or optionally substituted C₂-C₂₀ alkynyl. In various embodiments, A is attached to R⁵ via a single bond. For example, A is —NH— and -A-R⁵ may be —NH—R⁵. In various embodiments, A is attached to R⁵ via a double bond. For example, A is —N═ and -A=R⁵ may be —N═R⁵.

In various embodiments, B is present as a 5-membered or 6-membered heterocyclic ring having at least one N heteroatom in the ring. In various embodiments, a heterocyclic ring having at least one N heteroatom is also understood to mean that at least one C atom in the ring is replaced with one nitrogen containing substituent/group selected from N or NR^{c}. In various embodiments, B is present as a 5-membered or 6-membered heterocyclic ring having up to three C atoms in the ring optionally replaced with heteroatoms selected from the group consisting of N (or NR^{c}), O and S.

In various embodiments, B is a 5-membered heterocyclic ring represented by the following structure:

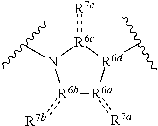

wherein

R^{6a}, R^{6b}, R^{6c} and R^{6d} are each independently selected from the group consisting of C, CR^{a}, CR^{a}R^{b}, N, NR^{c}, O or S, wherein R^{a}, R^{b}, and R^{c} are each independently selected from the group consisting of H, optionally substituted alkyl, optionally substituted alkenyl and optionally substituted alkynyl; and

R^{7a}, R^{7b} and R^{7c} are optionally present as ═O, ═S, —F, —Cl, —Br, —I, ═CR^{a}R^{b}, —CR^{a}R^{b}R^{c}, —OH, —SH, —NH₂ or ═NR^{c}.

In various embodiments, the dotted lines between R^{6a} and R^{7a}, R^{6b} and R^{7b}, and R^{6c} and R^{7c} represent optional single or double chemical bonds. In various embodiments, R^{6a} is attached to R^{7a} via a single bond, R^{6b} is attached to R^{7b} via a single bond, and/or R^{6c} is attached to R^{7c} via a single bond. For example, when R^{6a} is C and R^{7a} is —Br, then R^{6a}R^{7a} is C—Br. In various embodiments, R^{6a} is attached to R^{7a} via a double bond, R^{6b} is attached to R^{7b} via a double bond, and/or R^{6c} is attached to R^{7c} via a double bond. For example, when R^{6a} is C and R^{7a} is ═O, then R^{6a}R^{7a} is C═O.

In various embodiments, B is selected from the group consisting of succinimide (or pyrrolidine-2,5-dione), thiosuccinimide (or 5-thioxopyrrolidin-2-one), dithiosuccinimide (or pyrrolidine-2,5-dithione), pyrrolidine, pyrrole, pyrazolidine, maleimide (or pyrrole-2,5-dione), 1,2,3-triazole, 1,2,4-triazole and imidazole.

In various embodiments, only one of A and B is present in general formula (III). For example, when A is present in general formula (III), then B is absent from general formula (III), and vice versa.

In various embodiments, B is present while A is absent from general formula (III). In such embodiments, Y may be represented by general formula (IIIa):

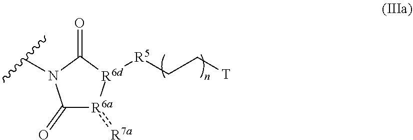

In various embodiments, A is present while B is absent from general formula (III). In such embodiments, Y may be represented by general formula (IIIb):

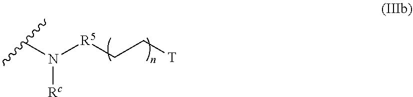

In various embodiments, both A and B are absent from general formula (III). In such embodiments, Y may be represented by general formula (IIIc):

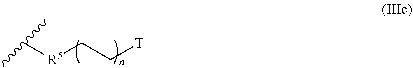

In various embodiments, R⁵ is selected from the group consisting of C₁-C₂₀ alkyl, C₂-C₂₀ alkenyl, C₂-C₂₀ alkynyl, C₁-C₂₀ alkoxy, C₁-C₂₀ alkoxyalkyl, C₂-C₂₀ alkylcarbonyl and C₃-C₂₀ alkylcarbonylalkyl. In various embodiments, R⁵ is straight or branched C₁-C₃ alkenyl. For example, R⁵ may be ethenyl, allyl or propenyl. In various embodiments, R⁵ is straight or branched C₁-C₃ alkyl. For example, R⁵ may be methyl, ethyl, n-propyl, 2-propyl, or isopropyl. In various embodiments, when Y is represented by general formula (IIIc), R⁵ is methyl.

In various embodiments, T is a terminal group selected from the group consisting of hydrogen and methyl.

In various embodiments, Y is selected from the following general formulae (IIIg), (IIIh) or (IIIi):

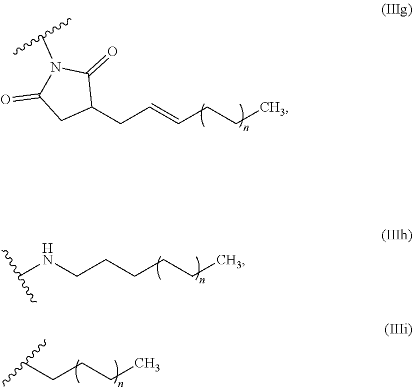

wherein n is from 10 to 350.

In various embodiments, Y comprises one or more of the following properties: inert; long shelf life; mechanical strength; impact resistant; thermal stability; elasticity; elastic recovery; smoothness; lightweight; low or non-toxicity; and miscibility, compatibility or affinity to lipophilic compound/polymer.

In various embodiments, Y is substantially devoid of polyalkylene glycol such as polyethylene glycol.

In various embodiments, the total molecular weight of general formula (II) is kept to no more than about 50,000, no more than about 45,000, no more than about 40,000, no more than about 35,000, no more than about 30,000, no more than about 25,000, no more than about 20,000, no more than about 15,000 or no more than about 10,000. It will be appreciated that copolymerisation may become inefficient when the total molecular weight of general formula (I) and (II) is too high.

In various embodiments, the molecular weight and/or length of the polymeric linker L is selected such that the overall molecular size of the repeating unit represented by general formula (I) is similar/comparable to the molecular size of the repeating unit represented by general formula (II). For example, if polyethylene (PE) having a molecular weight of 4,000 is selected as the choice of synthetic polymer for Y and peptide having a molecular weightof from about 400 to about 500 is selected as the choice of bioactive moiety X, then L may be designed to comprise a molecular weight of about 3,400. It will be appreciated that in various embodiments, it is the length of L that gets adjusted to match the molecular weight of general formula (I) to molecular weight of general formula (II). In other embodiments, the molecular weight and/or length of the polymeric linker L is selected such that the overall molecular size of the repeating unit represented by general formula (I) is different from the molecular size of the repeating unit represented by general formula (II).

In various embodiments, the molecular weight of general formula (I) is comparable/substantially similar with/to the molecular weight of general formula (II). In various embodiments, the molecular weight of general formula (I) does not differ from the molecular weight of general formula (II) by more than 30% of the molecular weight of general formula (II) or vice versa. For example, the molecular weight of general formula (I) may be at most about 30% more or at most 30% less than the molecular weight of general formula (II) or vice versa. The molecular weight of general formula (I) may not differ from the molecular weight of general formula (II) by more than about 30%, more than about 25%, more than about 20%, more than about 15%, more about 10%, more than about 5%, more than about 4%, more than about 3%, more than about 2%, or more than about 1% of the molecular weight of general formula (II) or vice versa. In various embodiments, the molecular weight of general formula (I) does not differ from the molecular weight of general formula (II) by more than about 20% of the molecular weight of general formula (II) or vice versa. For example, the molecular weight of general formula (I) may be at most about 20% more or at most 20% less than the molecular weight of general formula (II) or vice versa. Advantageously, as the bioactive moiety bearing repeating unit has a molecular size/weight/length that is similar to that of the polyethylene bearing repeating unit, the length of the bioactive moiety X is extended, thereby allowing X to be “visible”, available for binding to cells or accessible to its targeted physiological site for desired bioactivity, i.e. not buried in a sea/matrix of polyethylene chains. In other embodiments, the molecular weight of general formula (I) is not comparable/substantially similar with/to the molecular weight of general formula (II). In various embodiments, the molecular weight of general formula (I) differs from the molecular weight of general formula (II) by more than 30% of the molecular weight of general formula (II) or vice versa. For example, the molecular weight of general formula (I) may be about 30% more or 30% less than the molecular weight of general formula (II) or vice versa. The molecular weight of general formula (I) may differ from the molecular weight of general formula (II) by more than about 30%, more than about 35%, more than about 40%, more than about 45%, more about 50%, more than about 55%, more than about 60%, more than about 65%, more than about 70%, more than about 75%, or more than about 80% of the molecular weight of general formula (II) or vice versa.

In various embodiments, the molecular weight of general formula (I) is kept to no more than about 50,000, no more than about 45,000, no more than about 40,000, no more than about 35,000, no more than about 30,000, no more than about 25,000, no more than about 20,000, no more than about 15,000 or no more than about 10,000. In various embodiments, the molecular weight of general formula (I) is from about 100 to about 15,000, from about 200 to about 14,000, from about 300 to about 13,000, from about 400 to about 12,000, from about 500 to about 11,000, from about 1,000 to about 10,000, from about 1,500 to about 9,500, from about 2,000 to about 9,000, from about 2,500 to about 8,500, from about 3,000 to about 8,000, from about 3,500 to about 7,500, from about 4,000 to about 7,000, from about 4,500 to about 6,500, from about 5,000 to about 6,000 or about 5,500. In various embodiments, when X comprises longer peptides that contain more than 10 amino acids and the molecular weight of L is about 6,000, then the molecular weight of general formula (I) is greater than about 7,000. In various embodiments, when X comprises large peptides having molecular weight of about 5,000 or more and the molecular weight of L (e.g., polyethylene glycol) is about 6,000, then the molecular weight of general formula (I) can be up to about 12,000.

In various embodiments, the molecular weight of general formula (II) is kept to no more than about 50,000, no more than about 45,000, no more than about 40,000, no more than about 35,000, no more than about 30,000, no more than about 25,000, no more than about 20,000, no more than about 15,000 or no more than about 10,000. In various embodiments, the molecular weight of general formula (II) is from about 100 to about 15,000, from about 200 to about 14,000, from about 300 to about 13,000, from about 400 to about 12,000, from about 500 to about 11,000, from about 1,000 to about 10,000, from about 1,500 to about 9,500, from about 2,000 to about 9,000, from about 2,500 to about 8,500, from about 3,000 to about 8,000, from about 3,500 to about 7,500, from about 4,000 to about 7,000, from about 4,500 to about 6,500, from about 5,000 to about 6,000 or about 5,500.

In various embodiments, the total molecular weight of general formula (I) and general formula (II) is kept to about 300,000, no more than about 300,000, no more than about 200,000, no more than about 100,000, no more than about 90,000, no more than about 80,000, no more than about 70,000, no more than about 60,000, no more than about 50,000, no more than about 45,000, no more than about 40,000, no more than about 35,000, no more than about 30,000, no more than about 25,000, no more than about 20,000, or no more than about 15,000 to facilitate copolymerisation.

In various embodiments, L is hydrophilic. As L is adjustable, the hydrophilicity and/or swelling of the repeating unit represented by general formula (I) and also the overall hydrophilicity and/or swelling of the bioactive polyethylene copolymer may be adjusted as desired. Advantageously, the presence of L increases the hydrophilicity of the repeating unit represented by general formula (I) and also the overall hydrophilicity of the bioactive polyethylene copolymer. Even more advantageously, the presence of L increases the hydrophilicity and waxiness of the bioactive polyethylene copolymer, therefore softening the polyethylene chains which are highly hydrophobic and crystalline (for high molecular weight PE), making the copolymer less stiff after processing. In various embodiments, L allows adjustment of the overall copolymer's hydrophilicity to give water uptake. In various embodiments, L also allows adjustment of the overall copolymer's waxiness to give smoothness. It will be appreciated by a person skilled in the art that, bioactive moieties and polyethylene are typically mutually incompatible as the individual bioactive moiety is generally hydrophilic while polyethylene is generally hydrophobic. With the use of L in repeating unit represented by general formula (I), it is advantageously shown that a balance may be achieved between the hydrophilicity (of the bioactive component) and the hydrophobicity (of the synthetic component, i.e. polyethylene) to increase their compatibility with each other.

In various embodiments, L is amorphous. Advantageously, the presence of L increases the amorphousness and/or decreases the crystallinity of the bioactive polyethylene copolymer, making the copolymer useful for crafting softer or less stiff plastics.

In various embodiments, L is a heteroalkylene having at least 20 carbon atoms, at least 30 carbon atoms, at least 40 carbon atoms, at least 50 carbon atoms, at least 60 carbon atoms, at least 70 carbon atoms, at least 80 carbon atoms, at least 90 carbon atoms, at least 100 carbon atoms, at least 150 carbon atoms, at least 200 carbon atoms, at least 250 carbon atoms or at least 300 carbon atoms. In various embodiments, L is C₂₀-C₃₀₀ heteroalkylene or a heteroalkylene having from 20 carbon atoms to 300 carbon atoms.

In various embodiments, L has a number average molecular weight of between about 500 and about 7,000. L may have a number average molecular weight of about 600, about 700, about 800, about 900, about 1,000, about 1,500, about 2,000, about 2,500, about 3,000, about 3,500, about 4,000, about 4,500, about 5,000, about 5,500, about 6,000, about 6,500 or about 7,000. In various embodiments, the number average molecular weight of L is from about 1,000 to about 6,000.

In various embodiments, the heteroatom in L is O. In various embodiments, L is polyalkylene glycol. In various embodiments, L is poly(C₂-C₄ alkylene glycol). L may be selected from the group consisting of polyethylene glycol (PEG), polypropylene glycol (PPG), polytetramethylene glycol (PTMG), polybutylene glycol (PBG) and the like. Advantageously, the use of a polyalkylene glycol such as PEG can increase hydrophilicity of the macromonomer and the resultant copolymer. In various embodiments, the polyalkylene glycol such as PEG are used as spacers, linkers or linking groups in the overall polymers, instead of as terminal groups.

In various embodiments, L is polyalkylene glycol having at least about 10 repeating units, at least about 15 repeating units, at least about 20 repeating units, at least about 21 repeating units, at least about 22 repeating units, at least about 23 repeating units, at least about 24 repeating units, at least about 25 repeating units, at least about 30 repeating units, at least about 40 repeating units, at least about 50 repeating units, at least about 60 repeating units, at least about 70 repeating units, at least about 80 repeating units, at least about 90 repeating units, at least about 100 repeating units, at least about 150 repeating units, at least about 200 repeating units, or at least about 250 repeating units. In various embodiments, L comprises from about 10 monomers/repeating units to about 250 monomers/repeating units. Unlike conventional polymers which uses a short PEG chain, embodiments of the bioactive polyethylene copolymer disclosed herein incorporate a long polyalkylene glycol chain of at least 20 repeating units at L.

In various embodiments, L is selected from the group consisting of PEG₅₀₀, PEG₆₀₀, PEG₇₀₀, PEG₈₀₀, PEG₉₀₀, PEG₁₀₀₀, PEG₁₁₀₀, PEG₁₂₀₀, PEG₁₃₀₀, PEG₁₄₀₀, PEG₁₅₀₀, PEG₂₀₀₀, PEG₂₅₀₀, PEG₃₀₀₀, PEG₃₅₀₀, PEG₄₀₀₀, PEG₄₅₀₀, PEG₅₀₀₀, PEG₆₀₀₀ and mixtures thereof.

In various embodiments, X is coupled to the poly(norbornene dicarboximide) backbone through a carboxylic acid functionality in the following arrangement: —R¹-L-NR³—C(═O)—X. Advantageously, by linking X through a carboxylic acid functionality, amine terminal group(s) in X is/are free up for delivering its bioactivity, therefore ensuring the bioavailability of X. It will be appreciated that as amine group(s) confer bioactivity, exhausting up amine groups in bioactive moieties for polymer binding may be undesirable.

In various embodiments, X is coupled to the poly(norbornene dicarboximide) backbone via peptide/amide linkage, i.e. —NR³—C(═O)—. Advantageously, the bioactive polyethylene copolymer disclosed herein is considerably stronger and/or stable than conventional polymers that contain ester linkages. Without being bound by theory, it is believed that amide linkages are stronger than ester linkages because ester linkages are more prone to hydrolysis, which may release bioactive moieties into the bloodstream, leading to a premature metabolism of bioactive moieties.

In various embodiments, one or more of H atoms in alkyl, alkenyl, alkynyl, alkoxyalkyl, alkylcarbonyl and alkylcarbonylalkyl is/are optionally replaced by hydroxy, hydroxyalkyl, halogen, haloalkyl, cyano, cyanoalkyl and nitro.

In various embodiments, R¹ is selected from C₁-C₂₀ alkyl. The C₁-C₂₀ alkyl substituents may be straight or branched substituents selected from methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl or the like. R¹ may be straight or branched C₁-C₄ alkyl substituents. In various embodiments, the length of R¹ is the same as the length of a repeating unit in L. For example, if L is poly(butylene glycol), then R¹ is butyl. In another example, if L is poly(ethylene glycol), then R¹ is ethyl. It will be appreciated that in various embodiments, R¹ is carefully designed to match L.

In various embodiments, R³ is selected from H, C₁-C₂₀ alkyl, C₂-C₂₀ alkenyl or C₂-C₂₀ alkynyl.

In various embodiments, Z¹ and Z² are each independently selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl. The poly(norbornene) backbone may be selected from the group consisting of poly(norbornene-imide), poly(norbornene-dicarboximide), poly(norbornene) backbone is poly(5-norbornene-2,3-dicarboximide), poly(7-oxanorbornene), poly(oxanorbornene-imide), poly(oxanorbornene-dicarboximide) and the like. In various embodiments, Z¹ and Z² are each independently selected from CR^{a}R^{b}, O, NR^{c}, SiR^{a}R^{b}, PR^{a} or S, wherein R^{a}, R^{b}, and R^{c} are each independently selected from the group consisting of H, C₁-C₂₀ alkyl, C₁-C₂₀ alkenyl and C₁-C₂₀ alkynyl. In various embodiments, Z¹ is CH₂. In various embodiments, Z² is CH₂.

In various embodiments, X comprises a bioactive moiety selected from proteins, peptides, carbohydrates, therapeutic/drug molecules and derivatives thereof. In various embodiments, proteins, peptides, carbohydrates or therapeutic/drug molecules derivatives thereof include proteins, peptides, carbohydrates or therapeutic/drug molecules that are or have been optionally modified to contain one carboxylic acid terminal group. In some embodiments, the bioactive moiety contains only one carboxylic acid terminal group.

In various embodiments, the bioactive moiety comprises a monocarboxylic acid. Advantageously, the use of a bioactive moiety having a monocarboxylic acid terminal group avoids the possibility of an undesirable crosslinking which may otherwise occur if there is more than one carboxylic acid. In various embodiments therefore, the bioactive moiety X is substantially devoid of more than one carboxylic acid terminal group, for e.g., a dicarboxylic acid or tricarboxylic acid.

In various embodiments, X comprises protein or peptide. X may be a peptide sequence, laminin-derived peptide, integrin binding peptide, cell-penetrating peptide, collagen sequence, collagen mimics or collagen mimic peptides or collagen fragment. In various embodiments, X comprises from 2 to 50 amino acid residues, from 2 to 40 amino acid residues or from 2 to 20 amino acid residues in any sequence. In various embodiments, X comprises 50 amino acid residues, 40 amino acid residues, 30 amino acid residues, 25 amino acid residues, 20 amino acid residues, 15 amino acid residues, 10 amino acid residues, 9 amino acid residues, 8 amino acid residues, 7 amino acid residues, 6 amino acid residues, 5 amino acid residues, 4 amino acid residues or 3 amino acid residues in any sequence. The amino acid residues may be selected from the group consisting of glycine, alanine, valine, leucine, isoleucine, methionine, proline, phenylalanine, tryptophan, asparagine, glutamine, glycine, serine, threonine, serine, asparagine, glutamine, tyrosine, cysteine, lysine, arginine, histidine, aspartic acid and glutamic acid. In various embodiments, X is a peptide sequence comprising 3 to 20 natural amino acids. X may be integrin binding peptide selected from the group consisting of arginine-glycine-aspartic acid (RGD), SRGDS and RGDS; laminin-derived peptide A5G81 (AGQWHRVSVRWGC); osteopontin derived peptides SVVYGLR; and cell-penetrating/antimicrobial peptide selected from IRIK or (IRIK)₂ or (IKKI)₃. In various embodiments, X is a collagen sequence comprising 3 to 20 units of glycine (G), proline (P) and hydroxyproline (Hyp) in any sequence or permutation. X may be collagen fragment having a (PHypG)ₙ type sequence, (PGHyp)ₙ type sequence, (HypGP)ₙ type sequence, (HypPG)ₙ type sequence, (GHypP)ₙ type sequence, (GPHyp)ₙ type sequence or collagen mimic DGEA.

In various embodiments, X comprises carbohydrate. In various embodiments, X comprises monosaccharide, disaccharide, oligosaccharide or polysaccharide. In various embodiments, X comprises from 2 to 50 saccharide units, from 2 to 40 saccharide units, from 2 to 20 saccharide units or from 10 to 14 saccharide units. In various embodiments, X comprises 50 saccharide units, 40 saccharide units, 30 saccharide units, 25 saccharide units, 20 saccharide units, 15 saccharide units, 14 saccharide units, 13 saccharide units, 12 saccharide units, 11 saccharide units, 10 saccharide units, 9 saccharide units, 8 saccharide units, 7 saccharide units, 6 saccharide units, 5 saccharide units, 4 saccharide units or 3 saccharide units or 2 saccharide units. X may be heparin sulfate (HS) or glycosaminoglycans (GAGs). In various embodiments, X is heparin oligosaccharide selected from the group consisting of DP8, DP10, DP12, DP14 and DP16. In various embodiments, X is hyaluronic acid which is the simplest form of glycosaminoglycan (GAG).

In various embodiments, X is chemically coupled to the rest of general formula (I) via its hydroxy group. For example, when X is carbohydrate/saccharide, oxidation and/or reductive amination reactions may be performed on the carbohydrate's hydroxy for linking X to general formula (I). —CH₂OH on the saccharide may be oxidised to —C(═O)H, which subsequently undergoes reductive amination using the —NH₂ terminal on L to create a peptide linkage.

In various embodiments, X comprises a carbohydrate/saccharide that contained or has been modified to contain one carboxylic acid terminal group. Modification by one or more chemical reaction(s) such as oxidation may be performed on the carbohydrate/saccharide to create a carboxylic acid group. In various embodiments, modification is performed on a hydroxyl group that is originally present in the carbohydrate/saccharide. In various embodiments, —CH₂OH on the carbohydrate/saccharide is oxidized completely to —C(═O)OH, which subsequently reacts with a —NH₂ terminal on L to create a peptide linkage that links the carbohydrate/saccharide to the rest of general formula (I)—X—C(═O)—NH-L-. It will be appreciated, however, that no modification to the carbohydrate/saccharide may be required/necessary if a carboxylic acid is naturally present in the carbohydrate/saccharide.

In various embodiments, X comprises therapeutic/drug molecule. In various embodiments, X comprises antibiotic, antimicrobial, antibacterial, blood thinning agents or anti-inflammatory agents. X may be penicillin, amoxicillin, amphotericin, ciprofloxacin (CIF), atorvastatin, aspirin or aminoglycoside-based molecules selected from streptomycin, ribostamycin or gentamycin. It will be appreciated that X may be any therapeutic or drug molecule that contains a carboxylic acid group.

In various embodiments, X is chemically coupled to the rest of general formula (I) via one of its chemical moiety selected from the group consisting of —COOH, —CH₂OH, —CH₂NH₂ and ═CHNH₂. For example, —CH₂NH₂ or ═CHNH₂ on the drug molecule may be coupled to a small dicarboxylic acid before reacting with a —NH₂ terminal on L to create a peptide linkage that links the drug molecule to the rest of general formula (I): X—C(═O)—NH-L-.

In various embodiments, X comprises a therapeutic/drug molecule that contained or has been modified to contain one carboxylic acid terminal group. Modification by one or more chemical reaction(s) such as oxidation may be performed on the therapeutic/drug molecule to create a carboxylic acid group. In various embodiments, modification is performed on a hydroxyl group that is originally present in the therapeutic/drug molecule. For example, in various embodiments when X is ribostamycin or gentamycin, —CH₂OH on the drug molecule is oxidized completely to —C(═O)OH, which subsequently reacts with a —NH₂ terminal on L to create a peptide linkage that links the drug molecule to the rest of general formula (I): X—C(═O)—NH-L-. It will be appreciated, however, that no modification to the therapeutic/drug molecule may be required/necessary if a carboxylic acid is already present in the therapeutic/drug molecule.

In various embodiments, the bioactive moiety is or has been modified to contain one carboxylic acid terminal group. For example, if a carboxylic acid terminal group is absent in a carbohydrate or therapeutic/drug molecule, the carbohydrate or therapeutic/drug molecule may be modified to add a carboxylic acid at one of the carbohydrate or therapeutic/drug molecule terminals. The modification may comprise oxidation reaction(s) to convert a hydroxy group in the carbohydrate to carboxylic acid.

In various embodiments, the repeating unit represented by general formula (I) is in an amount of from about 1 molar % to about 100 molar %, from about 2 molar % to about 99 molar %, from about 3 molar % to about 98 molar %, from about 4 molar % to about 97 molar %, from about 5 molar % to about 96 molar %, from about 10 molar % to about 95 molar %, from about 15 molar % to about 90 molar %, from about 20 molar % to about 85 molar %, from about 25 molar % to about 80 molar %, from about 30 molar % to about 75 molar %, from about 35 molar % to about 70 molar %, from about 40 molar % to about 65 molar %, from about 45 molar % to about 60 molar %, or from about 50 molar % to about 55 molar % relative to the copolymer. In various embodiments, the repeating unit represented by general formula (I) is in an amount of from about 1 molar % to about 10 molar % relative to the copolymer. In various embodiments, the bioactive moiety is about 2 molar %, about 3 molar %, about 4 molar %, about 5 molar %, about 6 molar %, about 7 molar %, about 8 molar %, about 9 molar % or about 10 molar % of the bioactive polyethylene copolymer. In some embodiments, the repeating unit represented by general formula (I) is in an amount of not more than about 10 molar % relative to the copolymer. It will be appreciated that in some embodiments, bioactive moiety is relatively insoluble in a non polar solvent, thereby making it difficult to include more than 10% of general formula (I) in the copolymer without using a large excess of general formula (I) that is costly to produce.

In various embodiments, R² is selected from C₁-C₂₀ alkyl, C₂-C₂₀ alkenyl, C₂-C₂₀ alkynyl, C₁-C₂₀ alkoxyalkyl, C₂-C₂₀ alkylcarbonyl or C₃-C₂₀ alkylcarbonylalkyl. The C₁-C₂₀ alkyl substituents may be straight or branched substituents selected from methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl or the like.

In various embodiments, the ratio of the number of repeating units represented by general formula (I) to the number of repeating units represented by general formula (II) in the bioactive polyethylene copolymer is from about 1:1 to about 1:100, from about 1:2 to about 1:99, from about 1:3 to about 1:98, from about 1:4 to about 1:97, from about 1:5 to about 1:96, from about 1:6 to about 1:95, from about 1:7 to about 1:90, from about 1:8 to about 1:85, from about 1:9 to about 1:80, from about 1:10 to about 1:75, from about 1:15 to about 1:70, from about 1:20 to about 1:65, from about 1:25 to about 1:60, from about 1:30 to about 1:55, from about 1:35 to about 1:50, or from about 1:40 to about 1:45. In various embodiments, the ratio of the number of repeating units represented by general formula (I) to the number of repeating units represented by general formula (II) in the bioactive polyethylene copolymer is about 1:10, about 1:15, about 1:20, about 1:25, about 1:30, about 1:35, about 1:40, about 1:45 or about 1:50.

In various embodiments, the number of repeating units represented by general formula (I) in the copolymer is from about 10 to about 1,000. In various embodiments, the number of repeating units represented by general formula (II) in the copolymer is from about 10 to about 1,000.

In various embodiments, the bioactive polyethylene copolymer has a number average molecular weight (Mₙ) of from about 2,000 to about 300,000, from about 3,000 to about 200,000, from about 4,000 to about 150,000, from about 5,000 to about 100,000, from about 10,000 to about 90,000, from about 20,000 to about 80,000, from about 30,000 to about 70,000, from about 40,000 to about 60,000, or about 50,000.

In various embodiments, the bioactive synthetic copolymer has a polydispersity index (PDI) of from about 1.0 to about 10.0. In various embodiments, PDI of the bioactive synthetic copolymer is about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10.0. In various embodiments, the bioactive polyethylene copolymer has a polydispersity index (PDI) of from about 1.0 to about 3.0, from about 1.05 to about 2.95, from about 1.1 to about 2.9, from about 1.2 to about 2.8, from about 1.4 to about 2.6, from about 1.6 to about 2.4, from about 1.8 to about 2.2 or about 2.0. In various embodiments, the PDI of the bioactive polyethylene copolymer is no more than 1.50.

In various embodiments, the one or more repeating units represented by general formula (I) and the one or more repeating units represented by general formula (II) are designed to link to the poly(norbornene) backbone via at least covalent interactions. In various embodiments, each repeating unit represented by general formula (I) is covalently bonded to the poly(norbornene) backbone and/or each repeating unit represented by general formula (II) is covalently bonded to the poly(norbornene) backbone. Advantageously, as bioactive moieties (in general formula (I)) are covalently bonded to the bioactive polyethylene polymer, bioactivity is localized. In various embodiments, the bioactive moieties such as biomolecules do not leach out from the polymer, therefore preventing undesirable/unwanted side effects caused by biomolecules entering the circulatory system and/or reaching unintended parts of the body system. Embodiments of the bioactive polyethylene copolymer therefore overcome problems faced by conventional biomolecules that are administered as drugs which may metabolized prematurely before therapeutic effects are achieved. In various embodiments, the bioactive moieties such as drug molecules do not leach out into media which can escape into the environment in the event that disposal is improperly managed.

It will be appreciated that other interactions such as Van der Waals interactions may also be present within the copolymer.

In various embodiments, the bioactive polyethylene copolymer comprises a brush, bottlebrush, block, comb or graft-copolymer structure. In various embodiments, the repeating units may be randomly distributed/arranged within the polymer.

In various embodiments, the one or more repeating units represented by general formula (I) comprises two or more different types of bioactive moiety X. In various embodiments, the one or more repeating units represented by general formula (I) comprises 2, 3, 4, 5, 6, 7 or 8 different types of bioactive moiety X. For example, within a bioactive polyethylene copolymer, there may be repeating units represented by general formula (I) comprising peptide as X and repeating units represented by general formula (I) comprising carbohydrate as X. Advantageously, in various embodiments, the bioactive polyethylene copolymer imparts two or more different types of bioactivities.

In various embodiments, the bioactive polyethylene copolymer is a random polymer or a block copolymer. In some embodiments, the block polymer is a di-block or a triblock polymer. For example, the copolymer may have or is made up of two or three different polymer blocks. In some embodiments, the multi-block copolymer comprises more than three polymeric blocks. The blocks may be randomly distributed/arranged within the polymer.

In various embodiments, the bioactive polyethylene copolymer is selected from one of the following: (SA-t-PE)ₚ-[(GPHyp)₃]_{q} copolymer comprising (GPHyp)₃ in general formula (I) and succinic acid-terminated polyethylene in general formula (II); SA-t-PE-RGD copolymer comprising RGD in general formula (I) and succinic acid-terminated polyethylene in general formula (II); and Amine-terminated PE-RGD copolymer comprising RGD in general formula (I) and amine-terminated polyethylene in general formula (II).

Advantageously, the bioactive polyethylene copolymer disclosed herein is highly customizable. Depending on the application that the bioactive polyethylene copolymer is intended, X with the desired biological activity may be selected to combine with Y having the desired physical attributions to eventually obtain the bioactive polyethylene copolymer with the desired repeating units represented by general formulae (I) and (II). For example, for stents that face biofouling issues, antimicrobial peptides can be incorporated into stent material to target biofouling. For joint implants (e.g. knee joint implants), polyethylene may be made more biocompatible by incorporating peptides that bind integrins for cartilage regeneration such as RGD peptide, or oligosaccharides that mimic cartilage environment and encourage chondrocyte binding. Some possible oligosaccharides may include hyaluronic acid fragments and sulfated saccharides.

In various embodiments, the bioactive polyethylene copolymer is blended with a base polymer for further use. In various embodiments, the base polymer is similar to or of the same type as Y used in general formula (II). For example, the base polymer may be polyalkylene/polyolefin such as polyethylene, ultra-high-molecular-weight polyethylene, polypropylene, and copolymers of ethylene and α-olefins. In various embodiments, a medical grade polymer is used for base material while low molecular weight polyethylene is used in the synthetic side chain of the bioactive polyethylene copolymer. Advantageously, embodiments of the bioactive polyethylene polymer allow for biomolecule to be blended into a base material that is similar to the polyethylene side arms of copolymer, without phase separation. Advantageously, in various embodiments, the hydrophilic PEG chain is well distributed in/within the copolymer structure, therefore giving a better blending result when synthetic polyethylene(PE)-peptide copolymer is blended with hydrophobic base materials.

In various embodiments, there is also provided the following copolymers:
A) the copolymers comprising general formula (III-1):

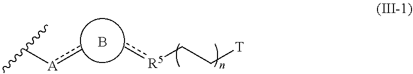

wherein

A is optionally present as N or NR^{c}, wherein R^{c} is independently selected from H, optionally substituted alkyl, optionally substituted alkenyl or optionally substituted alkynyl;

B is optionally present as a 5-membered or 6-membered heterocyclic ring having at least one N heteroatom in the ring;

R⁵ is selected from an optionally substituted alkyl, optionally substituted alkenyl, optionally substituted alkynyl, optionally substituted alkoxyalkyl, optionally substituted alkylcarbonyl or optionally substituted alkylcarbonylalkyl;

T is a terminal group selected from the group consisting of hydrogen and methyl; wherein the dotted lines represent optional chemical bonds;

n is from 10 to 350;
B) the copolymers prepared from a polyethylene macromolecule represented by general formula (VIII-1):

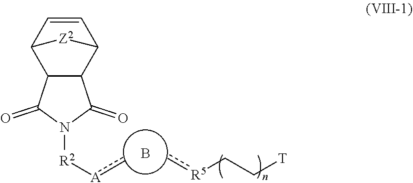

wherein

R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;

Z² is selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl;

A is optionally present as N or NR^{c}, wherein R^{c} is independently selected from H, optionally substituted alkyl, optionally substituted alkenyl or optionally substituted alkynyl;

B is optionally present as a 5-membered or 6-membered heterocyclic ring having at least one N heteroatom in the ring;

R⁵ is selected from an optionally substituted alkyl, optionally substituted alkenyl, optionally substituted alkynyl, optionally substituted alkoxyalkyl, optionally substituted alkylcarbonyl or optionally substituted alkylcarbonylalkyl;

T is a terminal group selected from the group consisting of hydrogen and methyl; wherein the dotted lines represent optional chemical bonds;

n is from 10 to 350; and
C) the copolymers derived from an intermediate described in a method of preparing a polyethylene macromolecule disclosed herein, the method comprising:
   (i) providing a dicarboxylic anhydride having general formula (IX):

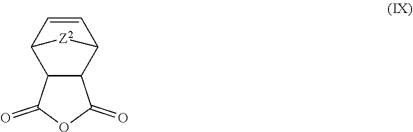

wherein Z² is selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl; and
(ii) reacting said dicarboxylic anhydride having general formula (IX) with an amine to obtain the polyethylene macromolecule, the amine is represented by general formula (X-1):

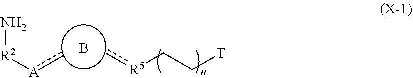

wherein

R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;

A is optionally present as N or NR^{c}, wherein R^{c} is independently selected from H, optionally substituted alkyl, optionally substituted alkenyl or optionally substituted alkynyl;

B is optionally present as a 5-membered or 6-membered heterocyclic ring having at least one N heteroatom in the ring;

R⁵ is selected from the group consisting of C₁-C₂₀ alkyl, C₂-C₂₀ alkenyl, C₂-C₂₀ alkynyl, C₁-C₂₀ alkoxy, C₁-C₂₀ alkoxyalkyl, C₂-C₂₀ alkylcarbonyl and C₃-C₂₀ alkylcarbonylalkyl;

T is a terminal group selected from the group consisting of hydrogen and methyl; wherein the dotted lines represent optional chemical bonds; and n is from 10 to 350.

In various embodiments, copolymers A)-C) possess the properties that are similarly desirable as the other copolymers disclosed herein.

In various embodiments, a bioactive polyethylene copolymer comprising general formula (III-1) possesses similarly desirable characteristics as a bioactive polyethylene copolymer comprising general formula (III).

In various embodiments, a bioactive polyethylene copolymer prepared from the polyethylene macromonomer represented by general formula (VIII-1) possesses similarly desirable characteristics as a bioactive polyethylene copolymer prepared from the polyethylene macromonomer represented by general formula (VIII).

In various embodiments, a bioactive polyethylene copolymer derived from the amine represented by general formula (X-1) possesses similarly desirable characteristics as a bioactive polyethylene copolymer derived from the amine represented by general formula (X).

Methods

There is provided a method of preparing a bioactive polyethylene copolymer, the method comprising: polymerizing one or more bioactive macromolecules represented by general formula (IV) with one or more polyethylene macromolecules represented by general formula (V) to obtain the bioactive polyethylene copolymer:

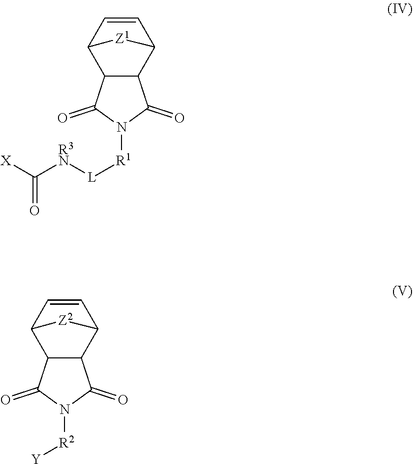

wherein

R¹ is optionally substituted alkyl;

R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;

R³ is selected from H, optionally substituted alkyl, optionally substituted alkenyl or optionally substituted alkynyl;

L is heteroalkylene;

X comprises a bioactive moiety selected from the group consisting of proteins, peptides, carbohydrates, therapeutic/drug molecules and derivatives thereof;

Y comprises polyethylene; and

Z¹ and Z² are each independently selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl.

Advantageously, in various embodiments, the method of preparing a bioactive polyethylene copolymer as disclosed herein is also a modular method for designing a bioactive polyethylene copolymer.

There is also provided a modular method of designing a bioactive polyethylene copolymer, the method comprising: selecting one or more macromolecules from a first module based on desired biological activity, the first module consisting of a library of norbornene-dicarboximide-containing bioactive macromolecules represented by general formula (IV) with known biological activities; selecting one or more macromolecules from a second module based on desired physical attributes, the second module consisting of a library of norbornene-dicarboximide-containing polyethylene macromolecules represented by general formula (V) with known physical attributes; and polymerizing the one or more macromolecules selected from the first module with the one or more macromolecules selected from the second module to obtain the bioactive polyethylene copolymer:

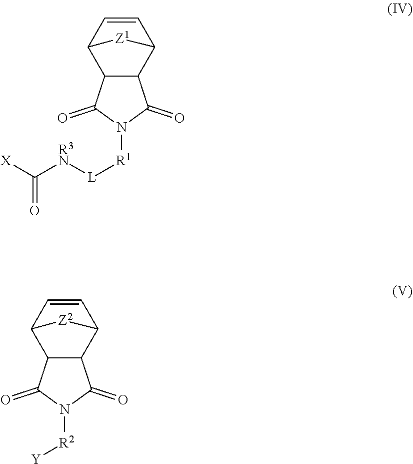

Advantageously, the methods disclosed herein allow rapid customization and quick development/construction of the bioactive polyethylene copolymer with the desired bioactivity and physical properties.

In various embodiments, the polymerization reaction comprises one or more olefin metathesis chain-growth polymerization step(s). The olefin metathesis chain-growth polymerization may be ring opening metathesis polymerization (ROMP). In various embodiments, the ROMP reaction occurs at the reactive moiety of the macromonomers, for e.g., at the olefins/alkene/C═C moieties. The ROMP may comprise a number of different approaches, including “arm-first” ROMP, “brush-first” ROMP, “graft-to” ROMP, “graft-from” ROMP, “graft-through” ROMP, or combinations thereof. Advantageously, ROMP allows quick development/construction of well-defined polyethylene polymers with the desired bioactivities. In various embodiments, depending on the targeted application, a biomolecule with the bioactivity of interest represented by general formula (I) may be chosen and copolymerized together with polyethylene represented by general formula (II) using ROMP.

In various embodiments, the polymerization reaction is performed in the presence of a polymerisation initiator/catalyst/promoter. In various embodiments, the polymerisation initiator/catalyst/promoter comprises a metal complex. The metal complex may be a ruthenium, molybdenum or tungsten complex. In various embodiments, ROMP is performed in the presence of a ruthenium complex. Advantageously, as compared to other transition metals (e.g., W and Mo), Ru is more stable in the presence of polar functional groups, thereby making Ru a suitable olefin metathesis catalyst for ROMP reactions that involve bioactive moieties selected from the group consisting of proteins, peptides, carbohydrates, therapeutic/drug molecules and derivatives thereof. In various embodiments, Ru is air-stable (i.e. stable in air) and thermally stable (i.e. stable at high temperatures) whilst being commercially available on a large scale, allowing ROMP to be carried out at elevated temperatures. The ruthenium complex may comprise a Grubbs catalyst selected from a first-generation Grubbs catalyst, second-generation Grubbs catalyst, Hoveyda-Grubbs' catalyst, a third-generation Grubbs catalyst or derivatives thereof.

In various embodiments, R¹, R², R³, L, X, Y, Z¹ and Z² contain one or more features and/or share one or more properties that are similar to those described above.

In various embodiments, the polymerization reaction comprises a) mixing one or more bioactive macromolecules represented by general formula (IV) with one or more polyethylene macromolecules represented by general formula (V) to obtain a solution; b) adding the catalyst to the solution from a); and c) precipitating the bioactive polyethylene copolymer.

In various embodiments, the polymerization reaction comprises mixing one or more bioactive macromolecules represented by general formula (IV) with one or more polyethylene macromolecules represented by general formula (V) in a ratio of from about 1:1 to about 1:10. In various embodiments, the one or more bioactive macromolecules represented by general formula (IV) is added to one or more polyethylene macromolecules represented by general formula (V) in a ratio of about 1:5.

In various embodiments, step a) and/or step b) is/are carried out or undertaken at a temperature in the range of from about 20° C. to about 180° C. In various embodiments, step a) and/or step b) is/are carried out or undertaken at a temperature just below the boiling point of the solvent used, for e.g., boiling point of 1,2-dichlorobenzene. The temperature(s) at which step a) and step b) is carried out may be independently selected from a temperature of about 20° C., about 25° C., about 30° C., about 35° C., about 40° C., about 50° C., about 60° C., about 70° C., about 80° C., about 90° C., about 100° C., about 110° C., about 120° C., about 130° C., about 140° C., about 150° C., about 160° C., about 170° C., or about 180° C. In various embodiments, high temperature reaction condition is required when working with higher molecular polyethylene (PE).

In various embodiments, step a) and/or step b) is/are carried out or undertaken for a time period in the range of from about 30 mins to about 3 days. The time period at which step a) and step b) is carried out may be independently selected from a time period of about 30 mins, 1 hour, 2 hours, 3 hours, 4 hours, 5 hours, 10 hours, 20 hours, 1 day, 2 days or 3 days.

In various embodiments, step a) and/or step b) is/are carried out in the presence of an organic solvent. In various embodiments, the organic solvent(s) for step a) and step b) is a non-polar solvent independently selected from the group consisting of benzene, toluene, dichlorobenzene and the like and combinations thereof. In various embodiments, non-polar solvents such as benzene and/or toluene may be used especially for PE-based materials. In various embodiments, the organic solvent used is the same for step a) and b). It is to be appreciated that the type of solvent used is dependent on the type of reactants used and is not limited to the above. In some embodiments, benzene can dissolve general formulae (I) and (II) at elevated temperatures as compared to toluene.

In various embodiments, step c) is/are carried out in a mixture of organic solvents. The mixture of organic solvents may contain one or more aprotic organic solvents and one or more protic organic solvents. In various embodiments, the mixture of organic solvents for step c) is selected from the group consisting of tetrahydrofuran (THF), benzene, toluene, acetonitrile (ACN), dichloromethane (DCM), dimethyl sulfoxide (DMSO), acetone, methyl ethyl ketone (MEK), ethyl vinyl ether, methanol, ethanol, butanol and the like and combinations thereof. It is to be appreciated that the type of solvent used is dependent on the type of reactants used and is not limited to the above. In various embodiments, ethyl vinyl ether is used to quench the catalyst added in step b). Only a few drops of ethyl vinyl ether may be added/used. In various embodiments, step c) is carried out by adding large quantity of protic solvent for e.g., methanol into the reaction mixture obtained from steps a) and b).

Advantageously, by carrying out polymerization with the carefully designed/controlled conditions described above, embodiments of the method disclosed herein have successfully overcome the widely varying and/or opposing properties of the individual components (e.g., L, X, Y components) to construct the bioactive polyethylene copolymer disclosed herein.

There is also provided a method of preparing a bioactive homopolymer, the method comprising: polymerising one or more bioactive macromolecules represented by general formula (IV) to obtain the bioactive homopolymer:

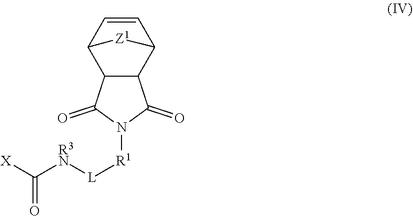

wherein R¹ is optionally substituted alkyl; R³ is selected from H, optionally substituted alkyl, optionally substituted alkenyl or optionally substituted alkynyl; L is heteroalkylene; X comprises a bioactive moiety selected from the group consisting of proteins, peptides, carbohydrates, therapeutic/drug molecules and derivatives thereof; and Z¹ is selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl.

There is also provided a method of preparing a polyethylene homopolymer, the method comprising: polymerising one or more polyethylene macromolecules represented by general formula (V) to obtain the polyethylene homopolymer:

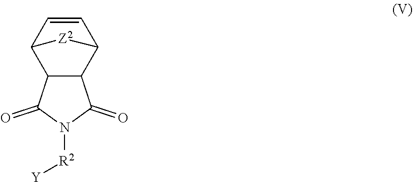

wherein R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl; Y comprises polyethylene; and Z² is selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl.

### Bioactive Macromolecule

There is also provided a bioactive macromolecule represented by general formula (IV) for preparing the copolymer disclosed herein:

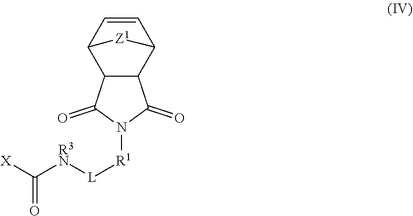

wherein

R¹ is optionally substituted alkyl;

R³ is selected from H, optionally substituted alkyl, optionally substituted alkenyl or optionally substituted alkynyl;

L is heteroalkylene;

X comprises a bioactive moiety selected from proteins, peptides, carbohydrates, therapeutic/drug molecules or derivatives thereof; and

Z¹ is selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl.

In various embodiments, R¹, R³, L, X and Z¹ contain one or more features and/or share one or more properties that are similar to those already described above.

In various embodiments, the bioactive macromolecule undergoes self-polymerization or co-polymerization. In various embodiments thereof, the bioactive macromolecule also behaves as a bioactive macromonomer.

In various embodiments, X is coupled to the norbornene dicarboximide through a carboxylic acid functionality in the following arrangement: —R¹-L-NR³—C(═O)—X. Advantageously, by linking X through a carboxylic acid functionality, amine terminal group(s) in X is/are free up for delivering its bioactivity, therefore ensuring the bioavailability of X. It will be appreciated that as amine group(s) confer bioactivity, exhausting up amine groups in bioactive moieties for polymer binding may be undesirable.

In various embodiments, X is coupled to the norbornene dicarboximide via peptide/amide linkage, i.e. —NR³—C(═O)—. Advantageously, the bioactive macromolecule disclosed herein is considerably stronger and/or stable than conventional macromolecules that contain ester linkages. Without being bound by theory, it is believed that amide linkages are stronger than ester linkages because ester linkages are more prone to hydrolysis, which may release bioactive moieties into the bloodstream, leading to a premature metabolism of bioactive moieties.

There is also provided a method of preparing a bioactive macromolecule disclosed herein, the method comprising: (i) providing a dicarboxylic anhydride having general formula (VI):

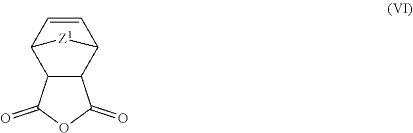

wherein Z¹ is selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl;
(ii) reacting said dicarboxylic anhydride having general formula (VI) with a diamine R⁴R³N-L-R¹—NH₂ to obtain an amine having general formula (VII):

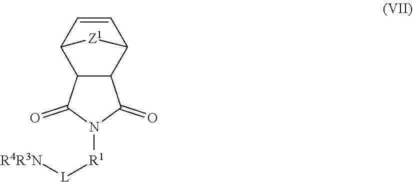

wherein R¹ is optionally substituted alkyl; R³ and R⁴ are each independently selected from H, optionally substituted alkyl, optionally substituted alkenyl or optionally substituted alkynyl, wherein at least one of R³ and R⁴ is H; L is heteroalkylene; Z¹ is selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl; and
(iii) reacting said amine having general formula (VII) with an acid-containing bioactive moiety X—C(═O)OH to obtain the bioactive macromolecule, wherein X comprises a bioactive moiety selected from the group consisting of proteins, peptides, carbohydrates, therapeutic/drug molecules and derivatives thereof.

In various embodiments, R¹, R³, L, X and Z¹ contain one or more features and/or share one or more properties that are similar to those described above.

In various embodiments, R³ and R⁴ are each independently selected from H, C₁-C₂₀ alkyl, C₂-C₂₀ alkenyl or C₂-C₂₀ alkynyl, wherein at least one of R³ and R⁴ is H.

In various embodiments, step (ii) comprises the use of diamine R⁴R³N-L-R¹—NH₂ for coupling X to norbornene dicarboxylic anhydride. The diamine used may be one that is commercially available. Advantageously, the method is a straightforward reaction and does not require a poly(ethylene glycol) aminocarboxylic acid which is commercially unavailable and synthetically challenging to make. In various embodiments therefore, the method does not require tedious multi-step and/or low yielding synthesis procedures. In various embodiments, the diamine R⁴R³N-L-R¹—NH₂ is used in slight excess to ensure that the amine having general formula (VII) does not become connected with norbornene dicarboximide on two ends, which would otherwise turn the diamine R⁴R³N-L-R¹—NH₂ into a linker instead of a terminating group.

In various embodiments, the diamine is a poly(ethylene glycol) diamine, wherein L is poly(ethylene glycol).

In various embodiments, the method further comprises, prior to step (iii), purifying the amine having general formula (VII) to isolate the product and/or remove impurities. In various embodiments, the step of purifying comprises washing with at least one of an acid or a base. The step of purifying may comprise washing with at least one of an acid or a base at least once, at least twice, at least thrice, at least four times, at least five times, at least six times, at least seven times or at least eight times to neutralise the amine having general formula (VII). In various embodiments, the step of purifying comprises double neutralisation steps. In one embodiment, the double neutralisation comprises a first step of washing with acid to remove unreacted diamine R⁴R³N-L-R¹—NH₂ and a second step of washing with base to neutralise the amine having general formula (VII). It will be appreciated that as the diamine R⁴R³N-L-R¹—NH₂ is basic, adding acid to said diamine will neutralise the diamine for removal from the amine having general formula (VII). It will also be appreciated that although the first step of washing with acid may protonate the amine having general formula (VII) at the amine terminal, the subsequent second step of washing with base or excess base converts the protonated form back into its free amine form. The acid used for the first neutralisation step may be selected from the group consisting of HCl, HNO₃, H₂SO₄ and H₃PO₄. The base used for the second neutralisation step may be selected from the group consisting of NaOH, KOH, NH₄OH and Ca(OH)₂. In various embodiments, the second neutralisation step comprises washing with base at least once, at least twice, at least thrice or at least four times to fully extract the amine having general formula (VII) for maximised yield. In one embodiment, the second neutralisation comprises washing with base twice. Without being bound by theory, it is believed that up to 30% of the protonated form of amine having general formula (VII) may reside in the aqueous phase during extraction. In various embodiments therefore, the step of washing with base comprises washing the aqueous phase once with base and washing the organic phase once with base in order to completely extract the amine having general formula (VII) from both the aqueous and organic phases. Advantageously, by using double neutralisation steps after coupling to obtain the free amine terminus, the method eliminates the need for any additional steps such as protection/deprotection step(s). It will be appreciated by a person skilled in the art that the use of diamine, particularly polyethyleneglycol diamine is extremely challenging and typically requires protection of one amine terminal in order to couple to a norbornene dicarboxylic anhydride. Indeed, in various embodiments, the polyalkylene glycol such as PEG are used as spacers, linkers or linking groups in the overall polymers, instead of as terminal groups. Thus, it may appear intuitive to consider protecting one amine terminal of a PEG diamine to couple it with norbornene dicarboxylic anhydride. The protecting group may then be removed to expose the amine terminus for further reactions. However, this would add extra steps to the reaction and hence may not be desirable. Embodiments of the present disclosure has managed to overcome this problem in the synthesis and purification steps by carrying out double neutralization steps after coupling to obtain the free amine terminus for further coupling to peptides.

### Polyethylene Macromolecule

There is also provided a polyethylene macromolecule represented by general formula (VIII) for preparing the copolymer disclosed herein:

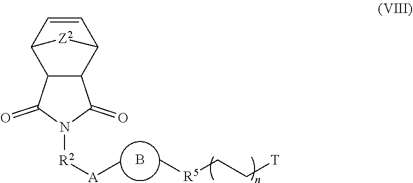

In various embodiments, the polyethylene macromolecule is represented by general formula (VIII-1):

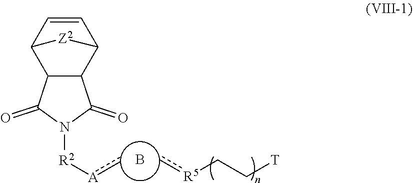

In various embodiments, A is present while B is absent from general formula (VIII-1). In such embodiments, the polyethylene macromolecule is represented by general formula (VIII-2):

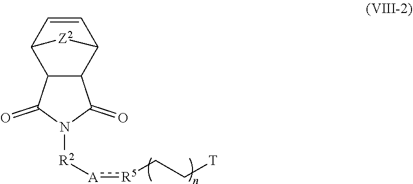

In various embodiments, the polyethylene macromolecule may be represented by one of the following general formula (VIIIa), (VIIIb) or (VIIIc):

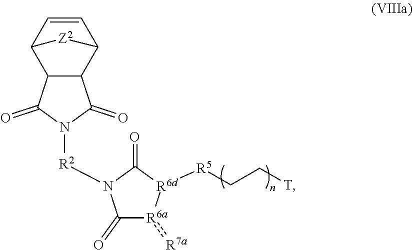

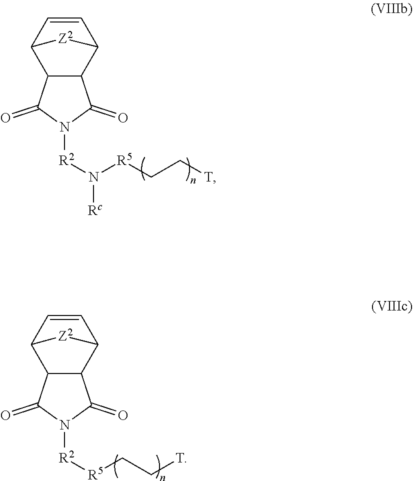

In various embodiments, Z², R², R^{6a}, R^{6d}, R^{7a}, R^{c}, A, B, R⁵ and T contain one or more features and/or share one or more properties that are similar to those already described above.

There is also provided a method of preparing a polyethylene macromolecule disclosed herein, the method comprising: (i) providing a dicarboxylic anhydride having general formula (IX):

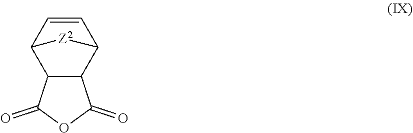

wherein Z² is selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl; and
(ii) reacting said dicarboxylic anhydride having general formula (IX) with an amine to obtain the polyethylene macromolecule, the amine is represented by general formula (X):

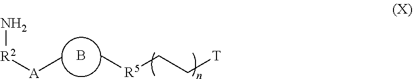

wherein
R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;
A is optionally present as NR^{c}, wherein R^{c} is independently selected from H, optionally substituted alkyl, optionally substituted alkenyl or optionally substituted alkynyl;
B is optionally present as a 5-membered or 6-membered heterocyclic ring having at least one N heteroatom in the ring;
R⁵ is selected from the group consisting of C₁-C₂₀ alkyl, C₂-C₂₀ alkenyl, C₂-C₂₀ alkynyl, C₁-C₂₀ alkoxy, C₁-C₂₀ alkoxyalkyl, C₂-C₂₀ alkylcarbonyl and C₃-C₂₀ alkylcarbonylalkyl;
T is a terminal group selected from the group consisting of hydrogen and methyl; and
n is from 10 to 350.

In various embodiments, the amine is represented by general formula

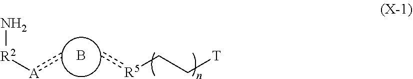

In various embodiments, A is present while B is absent from general formula (X-1). In such embodiments, the amine is represented by general formula (X-2):

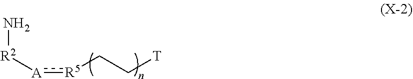

In various embodiments, general formula (X) may be represented by one of the following general formula (Xa), (Xb) or (Xc):

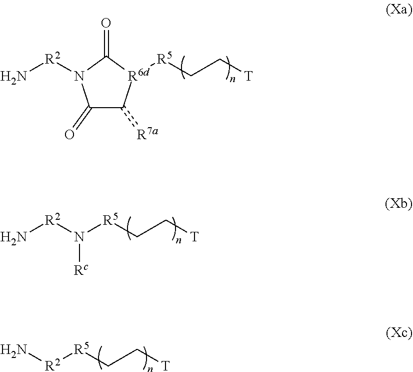

wherein
R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;
R⁵ is selected from the group consisting of C₁-C₂₀ alkyl, C₂-C₂₀ alkenyl, C₂-C₂₀ alkynyl, C₁-C₂₀ alkoxy, C₁-C₂₀ alkoxyalkyl, C₂-C₂₀ alkylcarbonyl and C₃-C₂₀ alkylcarbonylalkyl;
R^{6a} and R^{6d} are each independently selected from the group consisting of C, CR^{a}, CR^{a}R^{b}, N, NR^{c}, O or S, wherein R^{a}, R^{b}, and R^{c} are each independently selected from the group consisting of H, optionally substituted alkyl, optionally substituted alkenyl and optionally substituted alkynyl;
R^{7a} is optionally present as ═O, ═S, —F, —Cl, —Br, —I, ═CR^{a}R^{b}, —CR^{a}R^{b}R^{c}, —OH, —SH, —NH₂ or ═NR^{c}; and
T is a terminal group selected from the group consisting of hydrogen and methyl.

In various embodiments, the method further comprising, prior to step (ii), (a-i) providing a polyethylene having general formula (XIa) or (XIb):

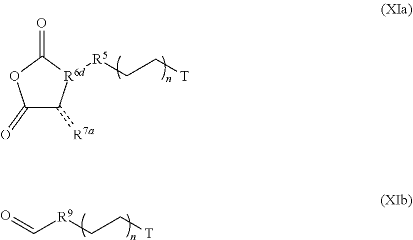

wherein
R^{6a} and R^{6d} are each independently selected from the group consisting of C, CR^{a}, CR^{a}R^{b}, N, NR^{c}, O or S, wherein R^{a}, R^{b}, and R^{c} are each independently selected from the group consisting of H, optionally substituted alkyl, optionally substituted alkenyl and optionally substituted alkynyl;
R^{7a} is optionally present as ═O, ═S, —F, —Cl, —Br, —I, ═CR^{a}R^{b}, —CR^{a}R^{b}R^{c}, —OH, —SH, —NH₂ or ═NR^{c};
R⁸ and R⁹ are each independently selected from the group consisting of C₁-C₂₀ alkyl, C₂-C₂₀ alkenyl, C₂-C₂₀ alkynyl, C₁-C₂₀ alkoxy, C₁-C₂₀ alkoxyalkyl, C₂-C₂₀ alkylcarbonyl and C₃-C₂₀ alkylcarbonylalkyl;
and
T is a terminal group selected from the group consisting of hydrogen and methyl; and
(b-i) reacting said polyethylene having general formula (XIa) or (XIb) with a diamine H₂N—R²—NH₂ or ammonia NH₃ to obtain the amine having general formula (X).

In various embodiments, -A=R⁵ linkage in general formula (X-1) or (X-2) is created by reacting a polyethylene having general formula (XIb) (for example PE-aldehyde or PE-CHO) with H₂N—R²—NH₂.

There is also provided a method of preparing a polyethylene macromolecule represented by general formula (VIII-1) or (VIII-2), the method comprising:
(i) providing a polyethylene having general formula (XIb):

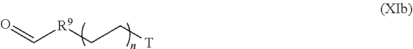

wherein
R⁹ is selected from the group consisting of C₁-C₂₀ alkyl, C₂-C₂₀ alkenyl, C₂-C₂₀ alkynyl, C₁-C₂₀ alkoxy, C₁-C₂₀ alkoxyalkyl, C₂-C₂₀ alkylcarbonyl and C₃-C₂₀ alkylcarbonylalkyl; and T is a terminal group selected from the group consisting of hydrogen and methyl; and
(ii) reacting said polyethylene having general formula (XIb) with a norbornene dicarboximide containing a pendant NH₂ to obtain the polyethylene macromolecule, the norbornene dicarboximide is represented by general formula (XII):

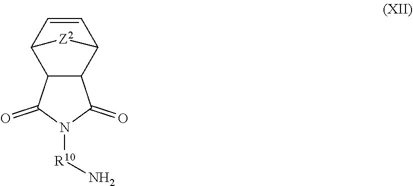

wherein
R¹⁰ is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl; and Z² is selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl.

In various embodiments, at least one of step (ii) and step (b-i) is performed in the presence of an organic solvent and/or a base.

In various embodiments, step (ii) and/or step (b-i) is/are carried out in the presence of an organic solvent. The organic solvent may be a non-polar solvent. In various embodiments, the organic solvent(s) for step (ii) and/or step (b-i) is an aromatic solvent such as toluene. Advantageously, in various embodiments, water removal from the condensation reaction is more efficient when toluene is used as a solvent. In various embodiments, non-polar solvents such as benzene, toluene, p-xylene, tetralin or decalin may be used. In various embodiments, the organic solvent(s) for step (ii) and/or step (b-i) is a halogenated solvent. The halogenated solvent may be a chlorinated solvent such as dichlorobenzene. In various embodiments, the organic solvent used is the same for step (ii) and (b-i). It is to be appreciated that the type of solvent used is dependent on the type of reactants used and is not limited to the above.

In various embodiments, step (ii) and/or step (b-i) is/are carried out in the presence of a base. The base may be an organic base selected from tertiary amine or pyridine. In various embodiments, the tertiary amine is selected from triethylamine.

In various embodiments, step (ii) and/or step (b-i) is/are carried out or undertaken at a temperature in the range of from about 80° C. to about 200° C. The temperature(s) at which step (ii) and step (b-i) is carried out may be independently selected from a temperature of about 80° C., about 90° C., about 100° C., about 110° C., about 120° C., about 130° C., about 140° C., about 150° C., about 160° C. about 170° C. about 180° C. about 190° C. or about 200° C.

In various embodiments, step (ii) and/or step (b-i) is/are carried out or undertaken for a time period in the range of from about 30 mins to about 3 days. The time period at which step (ii) and step (b-i) is carried out may be independently selected from a time period of about 30 mins, 1 hour, 2 hours, 3 hours, 4 hours, 5 hours, 10 hours, 20 hours, 1 day, 2 days or 3 days.

### Material Comprising Bioactive Polyethylene Copolymer

There is also provided a material comprising a copolymer as disclosed herein for use in medicine. In various embodiments, the material is part of or used on an apparatus selected from the group consisting of consumer care products such as diapers and sanitary products, and medical devices such as wound dressing, skin scaffold, bone scaffold, bone and bone marrow organoid scaffold, implants such as joint implants and cartilage implants, gut stents. For example, the material may be a scaffold, wound dressing or a medical device for tissue regeneration comprising the bioactive polyethylene copolymer disclosed herein. The material may be a material suitable for increasing biocompatibility of polyethylene used in medical devices through stimulation of collagen production. The material may be a material suitable for encouraging wound healing. The material may be a material for skin tissue regeneration (by incorporating bioactive moieties such as RGD or collagen). The material may be a knee joint implant for cartilage regeneration (by incorporating bioactive moieties such as hyaluronic acid or RGD). The material may be an antibacterial material suitable for use in wound dressings or tissue and serum handling devices. The material may be a material for increasing comfort in skin contact products such as diapers by incorporating collagen.

In various embodiments, the material is processed via electrospinning, melt extrusion, hot melt extrusion, injection moulding, fused filament fabrication or fused deposition modelling type three-dimensional printing, melt blowing and the like.

In various embodiments, the material or bioactive polyethylene copolymer is compatible with biological systems or parts of the biological systems without substantially or significantly eliciting an adverse physiological response such as a toxic reaction/response, an immune reaction/response, an injury or the like when used on/in the human or animal body. In various embodiments, the polymer is substantially devoid of materials that elicit an adverse physiological response.

There is also provided a method of accelerating/stimulating/promoting cell growth or tissue regeneration such as cartilage tissue or skin tissue regeneration, or wound healing, the method comprising administering/applying the bioactive active copolymer or material disclosed herein to a human or animal body.

There is also provided use of the bioactive polyethylene copolymer or material disclosed herein in the manufacture of a medicament for accelerating/stimulating/promoting cell growth or tissue regeneration, or wound healing such as, cartilage tissue or skin tissue regeneration.

For example, embodiments of the bioactive polyethylene copolymer or material disclosed herein may be useful in facilitating healing of wounds on diabetic patients (e.g. diabetes related injuries or wounds) such as diabetic foot ulcer. Advantageously, embodiments of the bioactive polyethylene copolymer or material disclosed herein may be useful to create dressings that possess the necessary stimulus within the material itself, to promote skin cell regeneration for enhanced recovery rates. Such materials would not only be useful for wound dressings but also in other medical devices that require enhanced tissue regeneration for improved healing outcomes. Some of such applications may include cartilage implants where collagen deposition around the implant by chondrocytes, is helpful to recovery from implantation.

There is also provided use of the bioactive polyethylene copolymer or material disclosed herein for biofilm eradication.

In various embodiments, the bioactive polyethylene copolymer is substantially devoid of stem cells and/or growth factor. In various embodiments, the bioactive polyethylene copolymer is non-biofouling.

In various embodiments, the bioactive moiety is directly chemically linked to the copolymer. In various embodiments, the bioactive moiety is not being encapsulated in the polymer matrix.

In various embodiments, the bioactive moiety (for e.g., peptide) is not connected to the norbornene dicarboximide via an amino butyric acid spacer.

In various embodiments, the bioactive moiety comprises structurally well-defined collagen with specific sequences. In various embodiments, the bioactive moiety is substantially devoid of animal derived collagen which have broad molecular weight distributions and/or ill-defined structures and/or known to illicit negative immune response in human body.

In various embodiments, polyethylene glycol is not used as a monomer on its own. For example, in various embodiments ethylene glycol units are not present in the copolymer/macromolecule as terminal groups.

Embodiments of the bioactive polyethylene polymer and/or methods disclosed herein do not involve any release of bioactive molecule such as drug molecule from the copolymer on activation methods such as photoactivation. Embodiments of the bioactive polyethylene polymer is substantially devoid of a photocleavable group.

### BRIEF DESCRIPTION OF FIGURES

FIG. 1 is a schematic diagram 100 of a bioactive polyethylene copolymer in accordance with various embodiments disclosed herein.

FIG. 2 shows the thermogravimetric analysis and differential scanning calorimetry (TGA-DSC) graphs of pure RGD peptide (“RGD(PURE)”), NBPEG₁₀₀₀RGD macromonomer (“NB PEG1000RGD”), NBPE homopolymer (“PE”) and succinic acid-terminated polyethylene (SAt-PE)-PEGRGD copolymer (“(RGD)ROMP”).

FIG. 3A is a graph showing the biocompatibility of PE-peptide based materials prepared in accordance with various embodiments disclosed herein, relative to a control. The results were obtained from cell viability tests on human keratinocytes cultured on a human de-epidermised dermis model. 3D printed sheets of bioactive PE blended with medical grade polypropylene were applied to the skin models for 24 and 48 h, where macromonomers of 6 peptides (SRGDS, RGDS, (IRIK)₂, (GPHyp)₃, DGEA and RGD) have been copolymerized with macromonomers of PE. Comparative examples are commercial dressings Allevyn (i.e. polyurethane foam) and Acticoat (i.e. silver-based polypropylene non-woven dressing). Control used include poly(norbornene dicarboximide) with PE and mPEG₁₀₀₀ as side chains.

FIG. 3B is a graph showing the biocompatibility of PE-peptide based materials prepared in accordance with various embodiments disclosed herein, relative to a control. The results were obtained from cell viability tests on a human de-epidermised dermis model, as ex vivo human skin models where macromonomers of 6 peptides (SRGDS, RGDS, (IRIK)₂, (GPHyp)₃, DGEA and RGD) have been copolymerized with macromonomers of PE, and the bioactive copolymers blended with polypropylene, extruded into filaments and 3D printed into sheets, then applied to the human skin models. Comparative examples are commercial dressings Allevyn (i.e. polyurethane foam) and Acticoat (i.e. silver-based polypropylene non-woven dressing). Control used include poly(norbornene dicarboximide) with PE and mPEG₁₀₀₀ as side chains.

FIG. 4 shows cross sectional hematoxylin and eosin (H&E) staining images of human skin samples obtained from preliminary ex vivo wound closure tests after 3 days of material application. Comparative example is commercial dressing Allevyn (i.e. polyurethane-based dressing). Control used include poly(norbornene dicarboximide) with PE and mPEGooo as side chains.

FIG. 5 shows cross sectional hematoxylin and eosin (H&E) staining images of human skin samples obtained from preliminary ex vivo wound closure tests after 3 days of application of PE-peptide based materials. The PE-peptide based materials are macromonomers of PE copolymerized with 6% of RGD macromonomer, and the final bioactive polyethylene copolymer blended with medical grade polypropylene at 0.1%, 1% and 3% blending ratios.

FIG. 6 shows the thermogravimetric analysis and differential scanning calorimetry (TGA-DSC) graphs of a bioactive amine-terminated polyethylene (PE)-RGD copolymer.

FIG. 7 is a graph showing the biocompatibility of PE-peptide based materials prepared in accordance with various embodiments disclosed herein, relative to commercial wound dressings Acticoat and Allevyn. The results were obtained from Hs27 human skin fibroblasts viability tests on PERGD copolymer blended with PLA at 1:4 ratio. PERGD copolymer blended with medical grade PLA was electrospun into nanofibers for biocompatibility tests with Hs27 human fibroblasts grown in DMEM w/10% FBS and 1% Pen/Strep.

### EXAMPLES

Example embodiments of the disclosure will be better understood and readily apparent to one of ordinary skill in the art from the following examples, tables and if applicable, in conjunction with the figures. It should be appreciated that other modifications related to structural, and chemical changes may be made without deviating from the scope of the invention. Example embodiments are not necessarily mutually exclusive as some may be combined with one or more embodiments to form new example embodiments. The example embodiments should not be construed as limiting the scope of the disclosure.

### Example 1: Modular Approach to Constructing Bioactive Polyethylene Copolymer

A general strategy for construction of bioactive macromonomers containing either peptides, carbohydrates or drug molecules has been developed. The simple 2-step synthesis allows for rapid buildup of a wide library of bioactive macromonomers of various chain length, allowing for quick development of synthetic polymers (i.e polyethylene) with desired bioactivities as required in targeted application. A modular approach to construct the desired polymers to suit various applications by matching the bioactive macromonomer with macromonomers of synthetic polymers with desired physical properties (i.e. polyethylene), is made possible using this library. Rapid polymer customization can thus be achieved.

A modular building block system to designing/constructing desired bioactive materials has been developed as shown in Scheme 1. Once the targeted medical application has been identified, a “plug and play” approach (Scheme 1) can be used to create the desired bioactive polyethylene material which not only possess therapeutic effects but also, necessary mechanical properties for easy storage and handling.

By using the modular approach, a macromonomer consisting of a bioactive molecule at the terminal of the monomer is created and can be copolymerized with other synthetic polymers (i.e. polyethylene) to create bioactive polyethylene copolymers with targeted bioactivities. The desired polymer is highly customizable using the strategy developed in accordance with various embodiments disclosed herein, by switching the bioactive molecule for any peptide or carbohydrate that bears a carboxylic acid group. This allows for rapid synthesis of bioactive polymers once a target application is identified. The bioactive polymers created can have properties ranging from skin cell regeneration, bone cell regeneration, antimicrobial activity, cartilage tissue regeneration, wound healing, collagen production, anti-inflammatory to cholesterol synthesis inhibition (for e.g., using atorvastatin as drug) and can be made to be mechanically tough, depending on the needs. The modular synthesis therefore makes application matching to polymer properties much simpler and effective.

### Example 2: Method of Preparing Bioactive Polyethylene Copolymer

The method of preparing a bioactive polyethylene copolymer in accordance with various embodiments disclosed herein involve creating macromonomers of the bioactive molecules and polyethylene separately and using ring opening metathesis polymerization (ROMP) techniques to link these otherwise mutually incompatible molecules together. The result is a brush polymer bearing both the bioactive molecule and the polyethylene for overall mechanical strength of the material (Scheme 2). By creating the macromonomers separately, the inventors are able to build a library of macromonomers with different properties for clinicians or medtech companies to choose from, and the material with desired therapeutic effects can be constructed easily and rapidly, to suit the targeted application. By creating the macromonomers separately, the inventors are also able to build a library of macromonomers and the eventual copolymers, for rapid testing of efficacy in the biomedical laboratory. Different combinations of these macromonomers (MMs) can also generate a library of well-defined brush copolymers containing different bioactive molecules for rapid screening of bioactivity in laboratory.

In the following examples, brush polymers containing pendant arms of polyethylene and bioactive molecules tethered on polyethylene glycol (PEG) moieties, have been created. Bioactive molecules may include biomolecules selected from peptide sequences of any combination of the 20 natural amino acids, from 3-20 amino acid residues, carbohydrates such as glycosaminoglycans or drug molecules containing a carboxylic acid terminal such as certain antibiotics. Biomolecules may also include collagen mimic peptides from 3-20 amino acid residues in any sequence, such as DGEA, (Gly-Pro-Hyp)₃ and (Pro-Hyp-Gly)₃. Depending on the application, the biomolecule with the bioactivity of interest can be chosen, and copolymerized together with polyethylene using the brush polymer technology as disclosed herein by ring opening metathesis polymerization.

The resultant polymer shows bioactivity of the biomolecule involved while having much better physical and mechanical properties for good material handling and processability.

The bioactive polyethylene copolymers can be subsequently blended with polymers similar to that on the pendant arms (e.g., polyethylene or polypropylene) to create bioactive materials for use in biomedical devices such as catheters, wound dressings, tissue scaffolds, plastic surgery implants, prosthetic parts, cartilage joint implants etc.

The synthetic route for preparing a bioactive polyethylene copolymer in accordance with various embodiments disclosed herein is illustrated in Scheme 2.

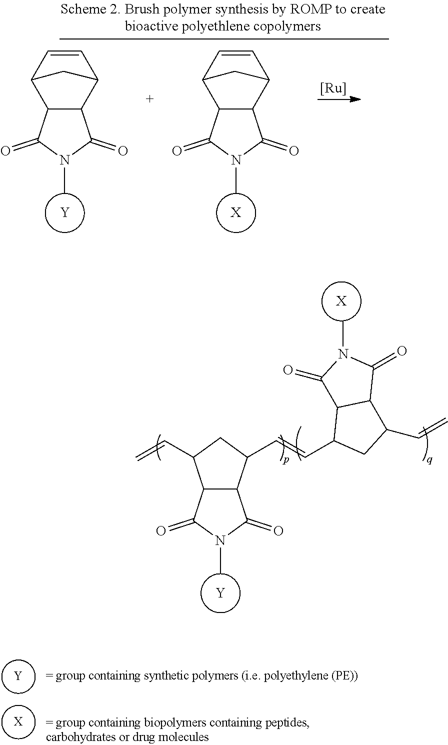

### Example 3: Bioactive Macromonomers and Method of Synthesis

A general strategy for the synthesis of bioactive macromonomers in accordance with various embodiments disclosed herein has been developed. Polyethylene glycol diamine of various chain lengths (e.g., M_{W}=1,000-6,000) are reacted with cis-norbornene-exo-2,3-dicarboxylic anhydride to create the main macromonomer body, i.e. macromonomer body containing norbornene dicarboximide and polyethylene glycol (NBPEG) (Scheme 3.1). Once NBPEG is created, various peptides, carbohydrates or drug molecules are then reacted with these NBPEG chains to create the bioactive macromonomer with desired therapeutic properties.

With the main body of macromonomer, any peptides, carbohydrates or drug molecules (R) can be used using the carboxylic acid terminus on the bioactive molecule by means of condensation reaction to form peptide/amide bonds between the amine group on NBPEG-NH₂ and carboxylic acid terminal of the carbohydrate or peptide (Scheme 3.2). Examples of drug molecules include antibiotics such as amoxicillin or ciprofloxacin. In various embodiments, R is copolymers of antimicrobial peptides (IRIK)₂ or (IKKI)₃; heparin oligosaccharides DP10, DP12, DP14, extracellular matrix peptides COL or RGD, where COL may be DGEA, (GPHyp)ₙ or (PHypG)ₙ, or (PGHyp)ₙ. In various embodiments, R is carbohydrates or drug molecules with a CO₂H group or peptide sequences of 3-20 amino acid residues, formed from 20 natural amino acids. In various embodiments, R is DP12, DP14, COL or RGD, where COL may be DGEA or (GPHyp)ₙ or any combinations of P, Hyp and G. Should the carbohydrate not possess a carboxylic acid group, modification of the carbohydrate to include one may be necessary. Alternatively, amine substitution reactions or reductive amination reactions can also be done on the carbohydrate's hydroxy or carbonyl groups.

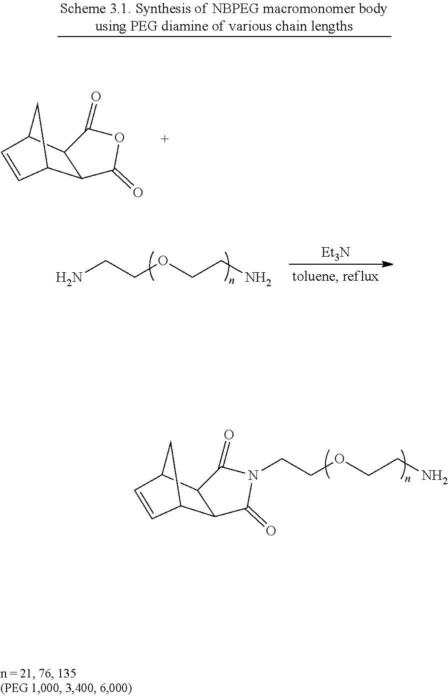

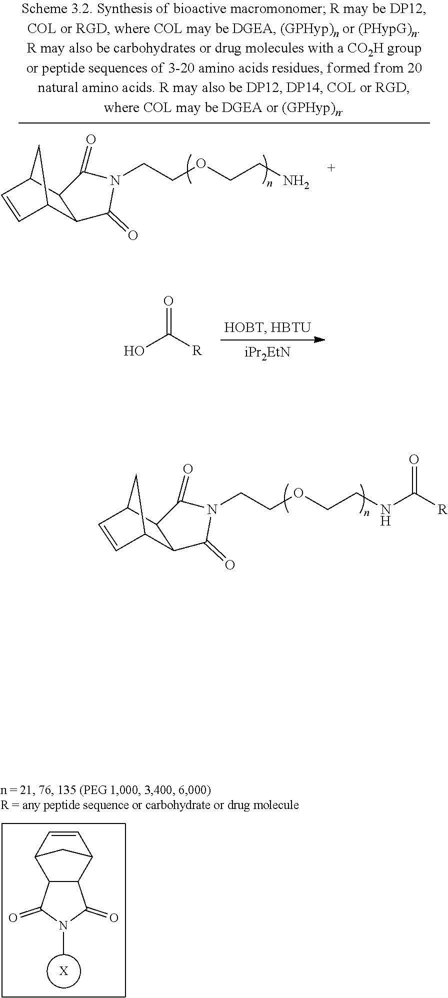

### Example 4: Polyethylene Macromonomers and Method of Synthesis

Polyethylene (PE) macromonomers may be created with a succinic acid terminus or a primary amine terminus.

To create PE macromonomers with a succinic acid terminus, vinyl-terminated polyethylene of low molecular weight and polydispersity is reacted with maleic anhydride to obtain a succinic acid-terminated polyethylene (SA-t-PE). This PE can then be reacted with hexamethylenediamine (HMDA) and finally, cis-norbornene-exo-2,3-dicarboxylic anhydride, to create the desired PE macromonomer (Scheme 4.1).

To obtain a primary amine terminus on polyethylene (PE) for macromonomer formation, hydroaminomethylation of vinyl terminated PE is carried out in a one pot, two-step process where it is first converted into a linear carbonyl via hydroformylation, followed by reductive amination in the presence of hexamethylenediamine (HMDA) (Scheme 4.2). Upon obtainment of the amine terminated PE, condensation with cis-norbornene-exo-2,3-dicarboxylic anhydride, affords the PE macromonomer (Scheme 4.3), which can be used in brush polymer formation with either itself or other macromonomers.

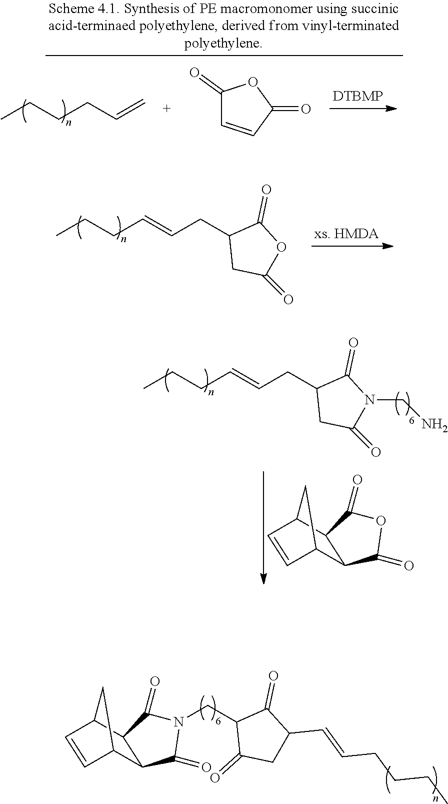

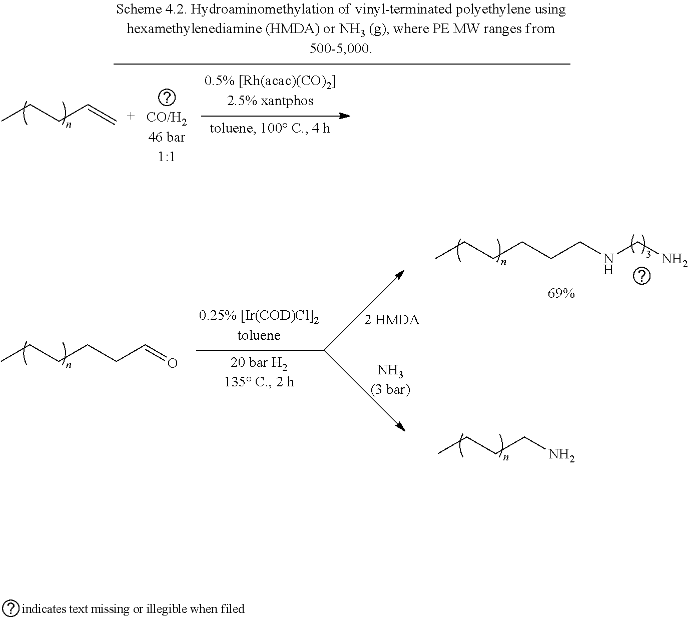

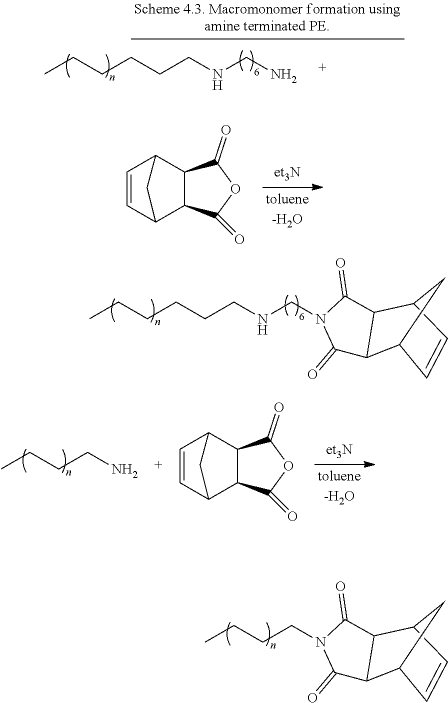

### Example 5: Ring Opening Metathesis Polymerisation Catalysts

With both the bioactive macromonomer and synthetic polymer (i.e. PE) macromonomer, the final bioactive polyethylene copolymer is prepared by ROMP using Grubbs type catalysts 1 (Scheme 5).

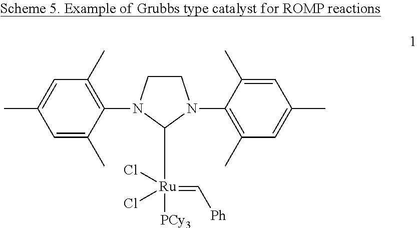

### Example 6: Bioactive Polyethylene Copolymers Examples

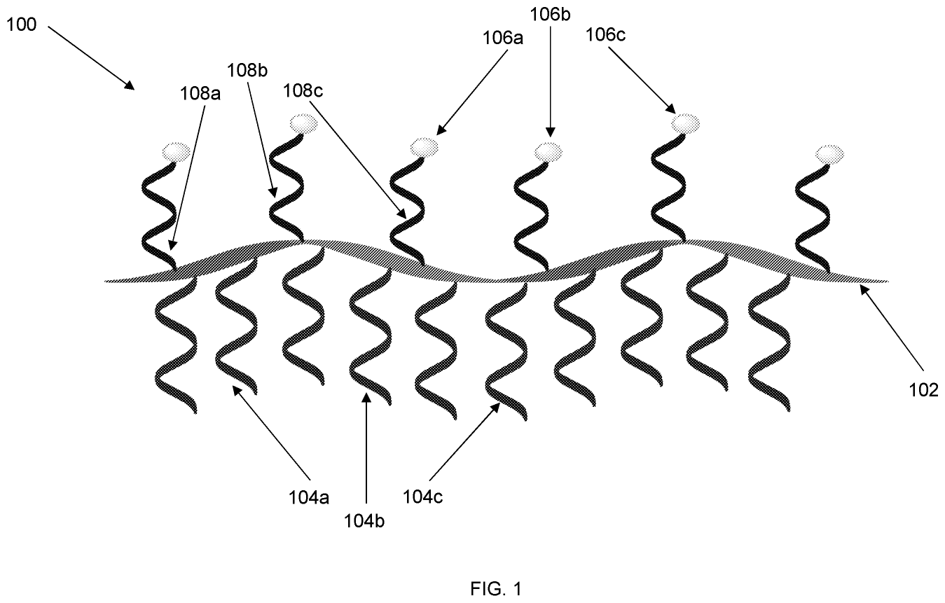

FIG. 1 shows a bioactive polyethylene copolymer 100 designed in accordance with various embodiments disclosed herein. The bioactive polyethylene copolymer 100 comprises a poly(norbornene dicarboximide) backbone 102, pendant arms of polyethylene 104a, 104b and 104c, and pendant arms of bioactive molecules 106a, 106b and 106c tethered on PEG chains 108a, 108b and 108c. As shown in the schematic diagram, the pendant arms are attached to the poly(norbornene dicarboximide) backbone 102. 106a, 106b and 106c may be the same or different types of bioactive moieties.

Examples of bioactive molecules include biomolecules selected from peptide sequences of 3-20 amino acid residues, formed from 20 natural amino acids, collagen mimic peptides from 3-20 amino acid residues in any sequence such as DGEA, (Gly-Pro-Hyp)₃ and (Pro-Hyp-Gly)₃, (Hyp-Pro-Gly)₃, (Gly-Hyp-Pro)₃, (Hyp-Gly-Pro)₃ and (Pro-Gly-Hyp)₃, carbohydrates such as glycosaminoglycans or drug molecules containing a carboxylic acid terminal such as certain antibiotics.

Collagen is a popular material in skin care and wound care industry due to their skin compatibility and reported ability to regenerate skin tissues. Yet, they are mild enough to prevent overactive tissue regeneration, potentially leading to cancer. The hydrophilicity of collagen itself makes it an attractive material for skincare products as the ability to preserve hydration in skin prevents dermatitis resulting from overly dry skin. Hence, the use of collagen fragments and collagen mimics may be used in PE to create polymers for blending into polypropylene base materials, for diaper manufacturing.

Naturally occurring biopolymers such as Hyaluronic Acid (HA) and Collagen (COL) have been shown to support cell growth and proliferation. Several studies have shown that HA improves the healing rate of diabetic foot ulcers (DFU) significantly. HA has been reported to enhance cartilage regeneration. Collagen gels or freeze dried collagen pads are available commercially for wound treatments. Integra, a collagen scaffold made of bovine collagen, is currently the gold standard for burns treatment. The ability of collagen to regenerate skin tissues and being a highly biocompatible material, makes it a very attractive material for wound healing materials.

Besides HA and COL, ArginylGlycylAspartic acid (RGD) peptides are yet another interesting peptide that has been reported to encourage both bone, cartilage and skin cell proliferation. The RGD sequence is the minimal binding domain for fibronectin, a high molecular weight glycoprotein of the extracellular matrix (ECM) that binds to ECM components such as collagen and fibrin. RGD peptide sequences are known to regulate cellular activity by interacting with cell-surface integrins which contribute to wound healing processes. Materials modified with RGD peptides have been reported to facilitate cell adhesion, spreading and wound healing. RGD also allows integrin binding for Transformational Growth Factor (TGF-β) activation which is required for regulation of cartilage development. Hence, RGD is an important peptide to consider in material development for wound healing and cartilage repair.

An example would be the development of a wound dressing material for treatment of chronic wounds where rapid skin reepithelization is required. RGD is a peptide sequence that is capable of binding integrins for cell attachment, migration and proliferation. Hence, macromonomer of RGD is created (Scheme 6a). As dressings are required to be inert with long shelf life and good mechanical strength, a polyethylene-bearing macromonomer is paired with this RGD-bearing macromonomer to create the eventual copolymer of RGD and PE (Scheme 6b). The material has been tested to be biocompatible and enhances reepithelization using human skin equivalent models.

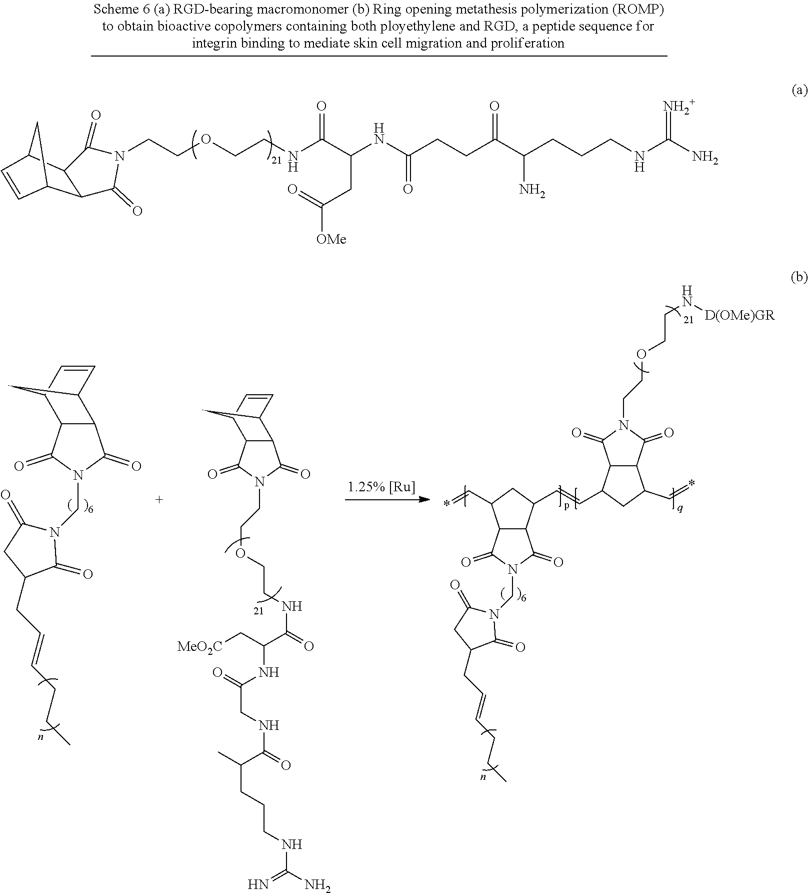

As RGD is a good integrin binder and bone growth factor binder, it can also be paired with PE macromonomer to create bioactive copolymers for use as substrate or scaffold material for bone and bone marrow organoid creation. Such a modular approach in building polymers allow the matching of different bioactive macromonomers with synthetic macromonomers to rapidly create mechanically strong therapeutic materials based on the targeted application.

RGD can be replaced with any peptide sequence via its acid terminal or any carbohydrate or any drug molecule such as amoxicillin or ciprofloxacin that has a carboxylic acid functional group.

For example, glycosaminoglycans (GAGs) such as heparin sulfate (HS) chains of between 5 to 10 disaccharide units may be used as the bioactive moiety. Without being bound by theory, it is believed that HS chains is active toward bone morphogenetic proteins (BMP), in particular, BMP-2, which is able to transdifferentiate myoblasts to osteoblasts. Without being bound by theory, it is believed that DP12, the HS fragment with hexa-disaccharide units, possesses the highest binding affinity for BMP-2.

Bioactive materials may also be created with collagen fragments and mimics. The bone is a mineralized collagenous tissue that remodels itself throughout one's lifecycle to adapt to mechanical stress and maintain the integrity of skeletal tissues. Current bone scaffolds are typically made of collagen sponges, occasionally mineralized with some calcium phosphate ceramics such as tricalcium phosphate or hydroxyapatite. The biocompatibility of collagen and its similarity to bone and cartilage tissues makes it an ideal scaffold material for bones and cartilage. Some possible collagen mimics that may be used include DGEA and collagen fragments bearing varying lengths of glycine, proline and hydroxyproline sequences. Without being bound by theory, it is believed that DGEA supports mesenchymal stem cell adhesion and differentiation to osteoblasts. Furthermore, without being bound by theory, it is believed that collagen also makes excellent skin scaffold materials since the extracellular matrix (ECM) is largely collagenous material. Other ECM peptides studied include laminin-derived peptide A5G81, which has been reported to facilitate wound healing in rats.

Besides tissue-regenerating biomolecules, cell-penetrating peptides such as (IRIK)₂ and (IKKI)₃ may also be used as the bioactive moiety, for incorporation into non-biofouling materials. Biofouling is a serious problem in biomedical devices such as catheters, gut stents and even wound dressings. The ability to target biofilm-forming bacteria such as P. Aeruginosa whilst not being toxic to human, makes such peptides attractive candidates for biomedical device materials.

Drug molecules such as antibiotics may also be incorporated into the brush polymers. Theoretically, any drug molecule with a carboxylic acid terminal would allow the creation of these bioactive synthetic polymers. Some of the drug molecules successfully polymerized include amoxicillin and ciprofloxacin. Brush polymers have been created, also for use in antimicrobial devices.

In summary, a general strategy has been developed to create bioactive macromonomers for rapid building of bioactive polyethylene copolymers by using a modular approach to polymer design and synthesis. Such a modular approach to therapeutic material synthesis allows for therapy customization to suit each patient's needs, hence moving closer to the ideal situation of personal medicine.

In summary, a series of polymers that bear polyethylene with pegylated biomolecules as side chains on a poly(norbornene dicarboximide) backbone have been developed, via ROMP technologies. The biocompatibility and skin cell viability of some of these polymers were also tested to demonstrate the materials' ability to withstand harsh material processing temperatures without loss in bioactivity. The general strategy presented here forms a method to create bioactive polyethylene copolymers for use as bioadditives in materials for biomedical devices where the bioadditive can be blended with a base polymer of similar type to the polymer side chain on the poly(norbornene dicarboximide) backbone. The polyethylene side chain helps make the biomolecule more compatible with the base synthetic polymer, allowing them to be blended together without phase separation. The formation of the brush polymer also allows the biomolecule to have better structural integrity as compared to the native biomolecule itself, which tends to be extremely hygroscopic, resulting in their poor handling and low processability, as a material.

### Example 7: Succinic Acid-Terminated Polyethylene (SA-t-PE)-Peptide Copolymers

### 7.1. (SA-t-PE)ₚ-[(GPHyp)₃]_{q} Copolymer

This example shows the development of a new strategy to incorporate collagen fragments or mimics into polyethylene by means of brush polymer synthesis, for use in consumer products such as diapers. The bioactive polyethylene created can be blended into medical grade polypropylene (PP) for non-woven fiber production which typically occurs at 220° C. by melt extrusion. Other peptides such as arginyl-glycyl-aspartic acid (RGD), known to enhance skin biocompatibility, can also be made using similar strategies.

Succinic acid-terminated polyethylene (SA-t-PE) was obtained according to the literature method (Macromolecules 2009, 42, 4356-4358; following the second example in Supporting Information).

### 7.1.1. PE Macromonomer

An amine handle was first created on SA-t-PE using hexamethylenediamine (HMDA), followed by connection of the aminated PE to a norbornene dicarboxylic anhydride linker (cis-norbornene-exo-2,3-dicarboxylic anhydride) to create the PE macromonomer (Scheme 7.1).

### 7.1.2. Peptide Macromonomer

Peptide macromonomer was prepared by reacting PEG diamine (MW 1,000-6,000) with cis-norbornene-exo-2,3-dicarboxylic anhydride on one amine end, followed by a second condensation reaction with a suitable peptide on the other amine end of PEG diamine (Scheme 7.2).

Macromonomers of collagen fragments such as (GPHyp)₃, (PGHyp)₃, collagen mimics such as DGEA and extracellular matrix peptides such as RGD, SRGDS, have also been prepared using this general strategy.

### 7.1.3. PE-Peptide Copolymer

Once both macromonomers have been synthesized, ring opening metathesis polymerization are carried out on the macromonomers using Grubbs' catalyst (Scheme 5, catalyst 1) to obtain the desired PE-peptide copolymer (Scheme 7.3).

The copolymer synthesized showed an average of 6% peptide incorporation in the polymer, despite a PE:peptide monomer ratio of 5:1. This is likely due to poor solubility of peptide macromonomer in benzene. Other non-polar solvents such as toluene are worst, with no more than 2% RGD incorporation. Polar solvents in general do not dissolve PE.

Upon synthesis, the polymers are checked for residual metals from Grubbs' catalyst using inductively coupled plasma mass spectrometry (ICP-MS) to ascertain that the metal content falls under 0.1 ppm. The FDA permissible inhalation limit for Ru in a class 2B medical device is 0.1 μg/g. For a 5 kg baby, this translates to 0.5 mg of Ru. Assuming each disposable diaper uses 10 g of non-woven PE-COL sheet as its cover and the blending ratio of PE-COL in PP is 3%, there is 0.3 g of PE-COL in the material. At 0.1 ppm Ru, the amount of Ru detected in 0.3 g of material is 0.03 μg. This is way below FDA limits for a class 2B device. For an application such as diaper, such a Ru limit is insignificant.

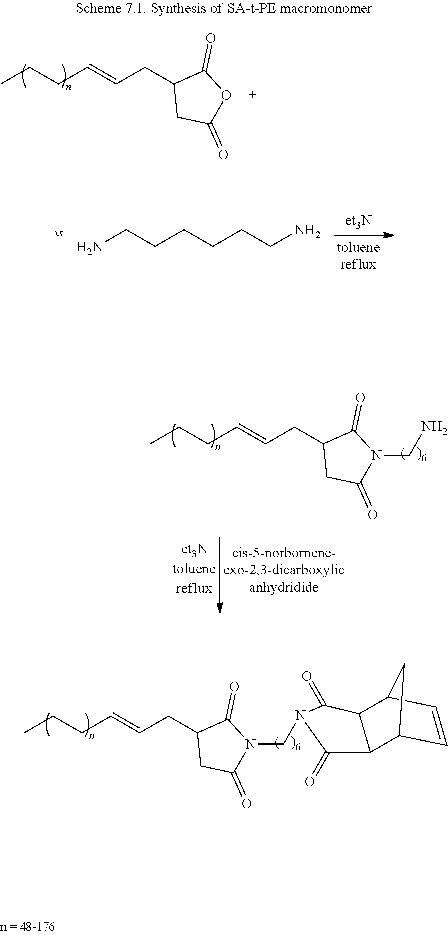

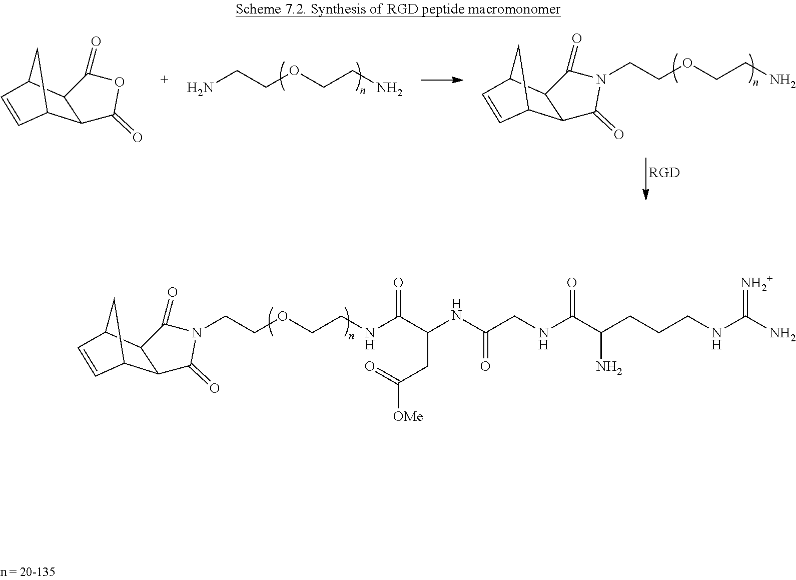

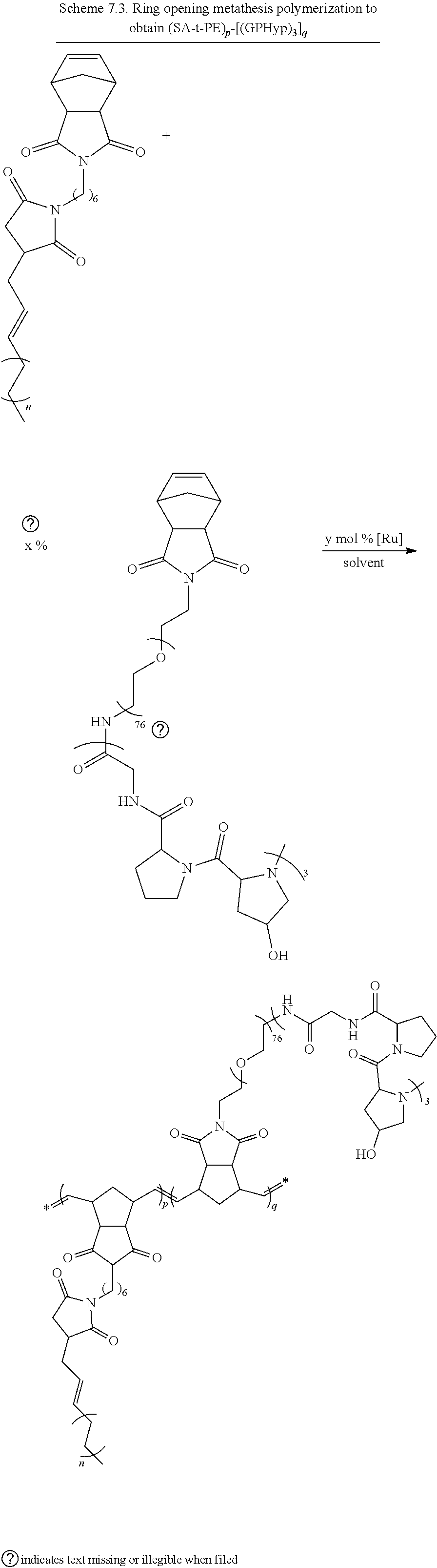

### 7.2. SA-t-PERGD Copolymer

The example shows the development of brush polymers with pegylated biomolecules such as RGD, HA and collagen fragments with polyethylene side chains, on a poly(norbornene dicarboximide) backbone, for applications in wound healing and cartilage repair.

Succinic acid-terminated polyethylene (SA-t-PE) was prepared by the literature method (Macromolecules 2009, 42, 4356-4358; following the second example in Supporting Information). The PE used to make SA-t-PE is in the MW range of 1,400-5,000 just right for use in grafting onto linker units such as norbornene, to create macromonomers that would be further polymerized into brush polymers comprising such PE side chains.

### 7.2.1. PE Macromonomer

An amine handle was first created on SA-t-PE using hexamethylenediamine (HMDA), followed by connection of the aminated PE to a norbornene dicarboxylic anhydride linker (cis-norbornene-exo-2,3-dicarboxylic anhydride) to create the PE macromonomer (Scheme 7.1).

### 7.2.2. Peptide Macromonomer

Peptide macromonomer was prepared by reacting PEG diamine (MW 1,000-6,000 depending on MW range of PE) with cis-norbornene-exo-2,3-dicarboxylic anhydride on one amine end, followed by a second condensation reaction with a suitable peptide on the other amine end of PEG diamine (Scheme 7.2).

### 7.2.3. PE-Peptide Copolymer

Once both macromonomers have been synthesized, ring opening metathesis polymerization are carried out on the macromonomers using Grubbs' catalyst (Scheme 5, catalyst 1) to obtain the desired PE-RGD copolymer (SA-t-PERGD) (Scheme 7.4).

The copolymer synthesized showed an average of 6% RGD incorporation in the polymer, despite a PE:RGD monomer ratio of 5:1. This is likely due to poor solubility of RGDPEGNB macromonomer in benzene. Other non-polar solvents such as toluene are worst, with no more than 2% RGD incorporation. Polar solvents in general do not dissolve PE.

Upon synthesis, the polymers are checked for residual metals from Grubbs' catalyst using ICPMS to ascertain that the metal content falls under 0.1 ppm. Assuming each cartilage implant (osteochondral plug) uses 1 g of PE to make, at 10% bioactive PE blending ratio, there is 0.1 g of bioactive PE in the implant material. At 0.1 ppm Ru, the amount of Ru detected in 0.1 g of bioactive RGD is 0.01 μg. This is way below FDA daily oral exposure limits of 100 μg/day or 1 μg/day by inhalation.

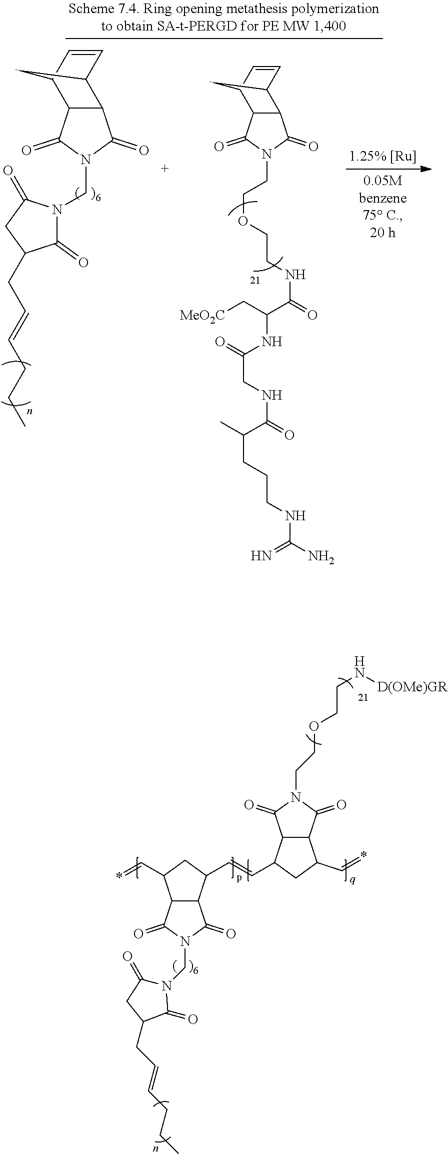

### 7.3. Thermal Stability

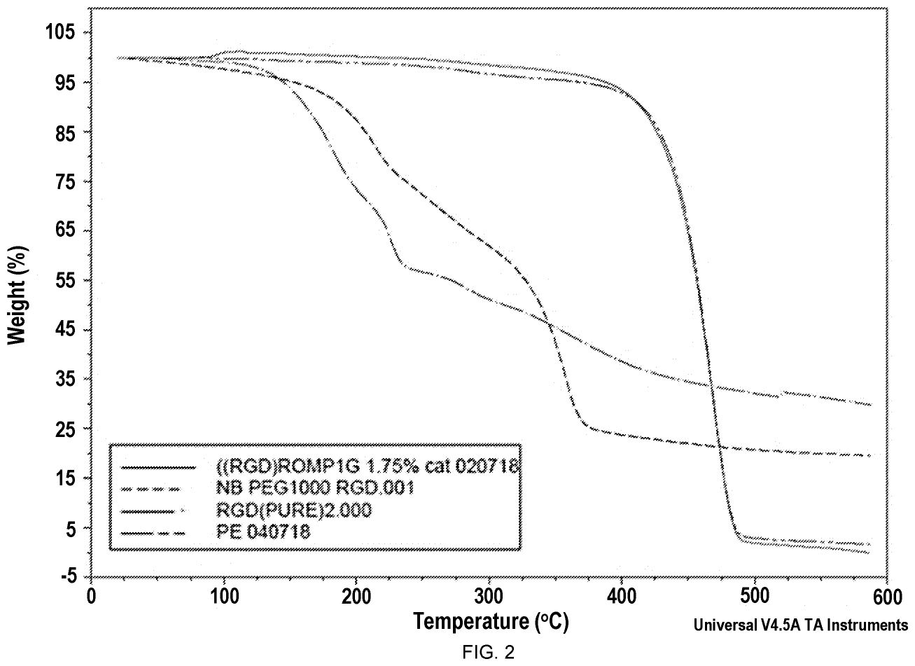

After ICP-MS, thermogravimetric analysis and differential scanning calorimetry (TGA-DSC) measurements were obtained on pure RGD, NBPEGRGD, NBPE homopolymer and PE-co-PEGRGD copolymer (PE-RGD) to ascertain thermal stability of the polymers. From the DSC curve, it is shown that the material undergoes a single-phase degradation/weight loss at >450° C. (467° C.), a temperature much higher than the typical melt processing temperature of PE or PP for either 3D printing or non-woven fiber production (FIG. 2). Hence, enhanced thermal stability of the material was demonstrated despite the incorporation of a biomolecule.

It was observed that pure RGD degrades at a temperature (150-250° C.) lower than its macromonomer NBPEG₁₀₀₀RGD (250-400° C.), and even much lower than its PE-RGD copolymer (400-500° C.). NBPEGRGD showed a two phase degradation (weight loss) where the first degradation was due to loss of RGD group, followed by decomposition of the PEG chain, from around 250° C. The PE-RGD copolymer only suffers from 50% weight loss at 466° C., allowing the use of most material processing methods such as melt extrusion or fused filament fabrication (FFF/FDM) to process the polymers into usable devices. The significant enhancement in thermal stability of RGD upon attachment to PEG followed by copolymerization with PE via ROMP on a norbornene dicarboximide backbone, is therefore evident here.

### 7.4. Biocompatibility

The bioactive PE was blended with medical grade polypropylene powder in varying ratios from 0.1-10% to create a formulation. The formulation was then melt extruded at 190° C. using a twin screw filament extruder to create Fused Filament Fabrication (FFF) printer quality filaments, which are then fed to the printer to create 2×2 cm² sheets of materials with 1.5×1.5 mm² pores, for ex vivo testing on human skin models.

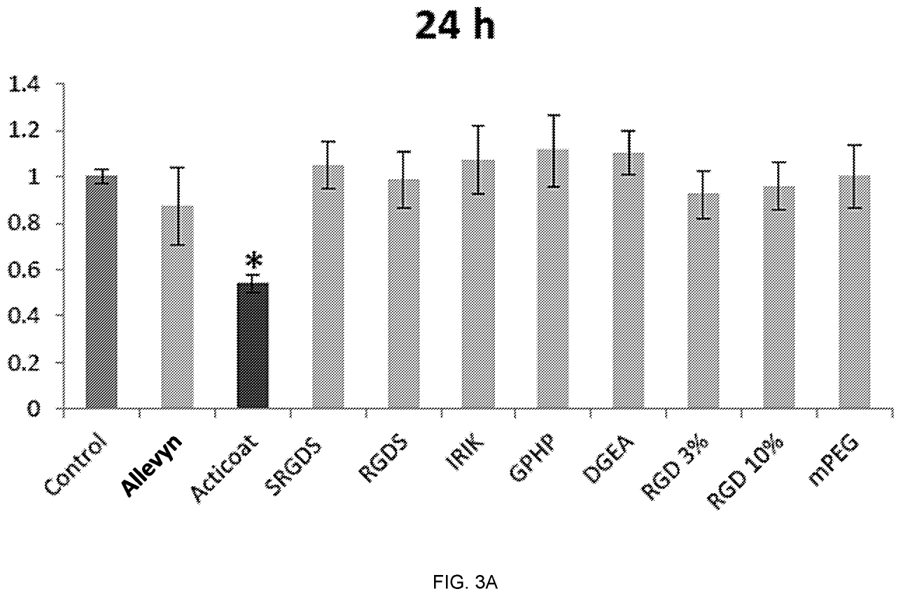

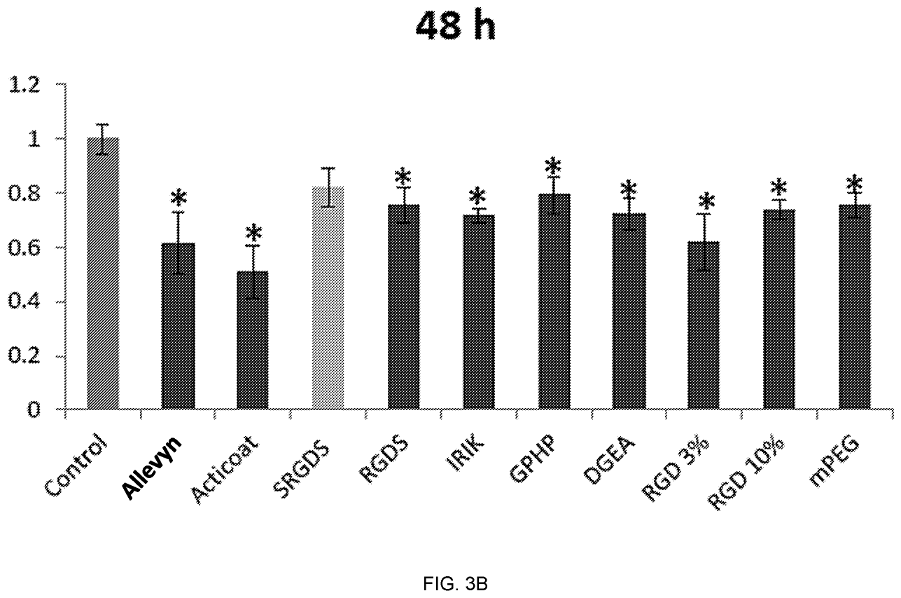

To demonstrate the bioactivity of the polymers designed in accordance with various embodiments disclosed herein, human skin testing was carried out using several of the polymers that had the peptides attached on the biomacromonomer. The bioactive synthetic polymer was blended with medical grade PP as base material and PP has a higher melting point of 179° C. The polymer blend was extruded at 220° C. into filaments using a filament extruder followed by FFF printing to give sheets with built in pores. The sheets were then tested on human skin models and clearly, the materials designed in accordance with various embodiments disclosed herein showed much better skin cell viability compared to commercial dressings such as Allevyn (polyurethane foam) and Acticoat (silver-based non-woven dressing) (FIG. 3A to FIG. 3B). These are two most commonly prescribed wound dressings in hospitals, for chronic wound patients. With these skin data, the efficacy of the bioactive synthetic polymers and their ability to withstand harsh material processing temperatures (220° C.) were demonstrated. Bioactivity of the biomolecule attached is not lost even at such temperatures, demonstrating the present disclosure's ability to create bioactive polymers that have high stability for material processing and bioefficacy for various treatments.

Biocompatibility tests were carried out in SRIS's human tissue lab using human dermis to reconstruct a skin model by regrowing an epidermis layer using keratinocytes obtained from the skin bank. The tests were conducted following standard reported protocols according to Topping, G. et al. (Primary Intention: The Australian Journal of Wound Management, 2006, 14(1), 14-21), the contents of which are fully incorporated herein by reference. Briefly, dressing materials were applied on the skin models with media provided to the skin to support skin viability. Dressings were removed at 24 h (FIG. 3A) and 48 h (FIG. 3B), and the skin samples stained with alamar blue, incubated for 90 min each, and the stains were analyzed by fluorescence spectroscopy to check for the amount of viable cells.

Copolymers were created using PE as the synthetic polymer and a range of peptides of different properties as the bioactive macromonomer, namely antimicrobial peptide: IRIK; collagen fragment (GPHyp)₃: GPHyp; collagen mimic: DGEA and integrin binding peptides: RGD, SRGDS and RGDS. The copolymers were subsequently blended with polypropylene and 3D-printed into sheets, before tested for cell viability and biocompatibility against commercially available wound dressing such as Allevyn and Acticoat.

From the ex vivo tests, it can be seen that the collagen-based materials, (GPHyp)₃ and DGEA both showed enhanced cell viability compared to controls, at 24 h. SRGDS also outperformed control slightly (FIG. 3A). At 48 h, both SRGDS and GPHyp retained good cell viability, demonstrating their biocompatibility with human skin (FIG. 3B). Even the bioactive PE made with an antimicrobial peptide such as (IRIK)₂, showed reasonable biocompatibility, compared to Acticoat, the commercial antimicrobial dressing.

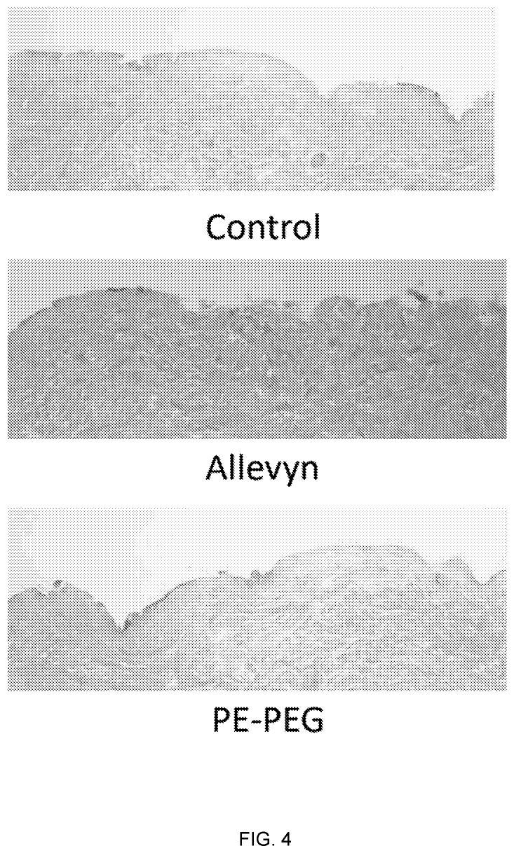

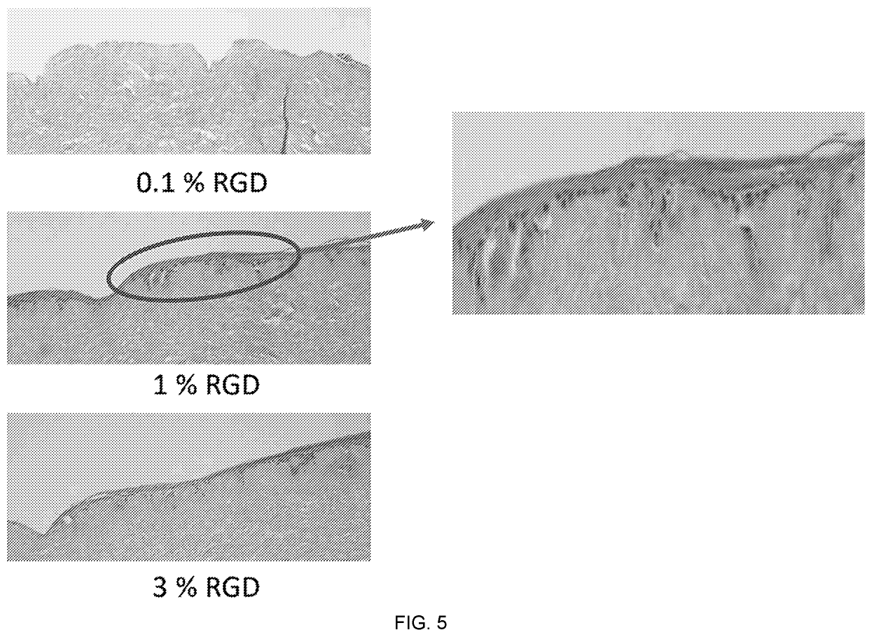

Cross sectional hematoxylin-stained and eosin-stained (H&E) images of human skin samples obtained from preliminary ex vivo wound closure tests after 3 days application of PE-peptide based materials are provided in FIG. 4 and FIG. 5. Comparative example is commercial dressing Allevyn (i.e. polyurethane-based dressing). Control used include poly(norbornene dicarboximide) with PE and mPEG₁₀₀₀ as side chains. The PE-peptide based materials are macromonomers of PE copolymerized with macromonomers of RGD and blended with polypropylene at ratios of 0.1%, 1% and 3% PE-RGD copolymer, respectively.

In short, copolymeric materials of PE with various peptides including collagen fragments, collagen mimics, antimicrobial peptides (IRIK)₂ and extracellular matrix peptides such as RGD and SRGDS using PEG and norbornene dicarboximide linkers have been prepared. The materials showed enhanced thermal stability as well as good biocompatibility data. In general, peptides of 3-20 amino acids in length in any sequence and oligosaccharides of up to 14 saccharide units, can be used in general.

### 7.5. Conclusion

Through the incorporation of pegylated peptides and collagen fragments into brush polymers containing polyethylene (PE) side chains, bioactive polyethylene was created for use in non-woven fibers for consumer products and polyethylene- or polypropylene-based medical devices. To create prototypes for testing, these polymers were blended into medical grade polypropylene as a powder and melt extruded at 220° C. into sheets using an FDM printer. The polymeric sheets have been tested ex-vivo on human skin models and demonstrated excellent biocompatibility with human skin. Other pegylated peptides have also demonstrated good biocompatibility upon incorporation into PE. As shown in the examples, the thermal stability of the peptides improved dramatically upon incorporation into PE-based brush polymers. Peptides of 3-20 amino acids in length in any sequence and oligosaccharides of up to 14 saccharide units, can be used in general.

Polymeric materials containing pegylated collagen fragments or peptides have been developed with polyethylene, as brush polymers using polynorbornene dicarboximide backbone. The polymers showed bioactivities of the peptides in terms of biocompatibility and enhanced cell viability, using ex vivo human skin models. At the same time, the polymers also showed improved thermal stability, as evident from the TGA-DSC curves which showed material weight loss of 50% only at 467° C. The polymers were melt extruded at 220° C. and showed no loss in bioactivity when tested on human skin, demonstrating the capabilities in developing bioactive polyethylene materials that have both thermal stability and bioactivity like that of collagen. The materials can be melt processed by conventional material processing methods, making them useful materials for consumer products such as diapers. They also demonstrated good mechanical properties like those of polyethylene.

Through the incorporation of pegylated biomolecules known to improve wound healing into polyethylene, novel materials were created that possess both bioactivity of the biomolecules used, as well as physical properties of polyethylene. Extracellular matrix peptides such as RGD, collagen and laminin-derived peptides such as A5G81, are known to enhance skin cell proliferation and wound healing. Brush polymers were created with these peptides on pegylated side chains, along with polyethylene side chains, on a polynorbornene dicarboximide backbone. The materials were then 3D printed using FFF printers into sheets and tested on human skin models. The ex vivo tests of the materials showed improved cell viability compared to commercial dressings as controls.

Brush polymers bearing polyethylene side chains and pegylated peptides have been created for use in tissue regeneration materials such as wound dressings and other medical devices. By incorporating peptides into polyethylene, bioactivity was created in an otherwise inert polyethylene. At the same time, the thermal stability of the peptides such as collagen fragments and RGD, were enhanced dramatically. The peptides also did not phase separate from PE. This allowed the bioactive PE to be blended into base polypropylene (PP), for non-woven fiber production in wound dressing manufacturing. The bioactive PE can also be blended into PE for creation of other biomedical devices such as joint implants and stents to improve recovery rates in patients receiving the implants as a result of more biocompatible PE being used.

### 7.6. Materials and Methods

### 7.6.1. General Procedure

Ring opening metathesis polymerization reactions and RGD macromonomer synthesis were carried out in a Vacuum Atmosphere glovebox under nitrogen atmosphere. SA-t-PE macromonomer was synthesized under ambient conditions. All the solvents used—anhydrous benzene and anhydrous methanol from Alfa Aesar, were used as purchased, in the glovebox. Grubbs second generation catalyst was purchased from Sigma Aldrich and all peptides (including RGD peptides) were purchased from Biomatik Inc. HMDA and PEG diamine were purchased from Alfa Aesar. All purchased reagents were used without further purification. Succinic acid terminated polyethylene (MW 15,000 and below) was prepared according to the literature (Macromolecules 2009, 42, 4356-4358; following the second example in Supporting Information).

¹H NMR spectra were recorded on a Jeol 500 MHz NMR spectrometer. GPC chromatogram were recorded on an Agilent Infinity II High Temperature GPC system equipped with 2*PLgel Mixed B columns (300×7.5 mm, particle size 10 μm) and 1*PLgel Mixed B guard column (50×7.5 mm). Eluent is TCB with 1 ml/min flow rate and oven temperature of 160° C. Polystyrene was used as calibration standard.

### 7.6.2. Synthesis of SA-t-PE Macromonomer (SA-t-PEHMDANB) for PE MW 1,400-5,000

SA-t-PE (6 mmol) was weighed into a 250 ml round bottomed flask (rbf) followed by addition of toluene (120 ml). HMDA (18 mmol) and triethylamine (6 mmol) were then added. The mixture was then stirred under reflux overnight with a dean stark trap connected, for water removal. The mixture was then cooled and concentrated, followed by addition of MeOH, to give a beige precipitate. The mixture was filtered and residue was washed with MeOH before drying in a vacuum oven overnight, to give SA-t-PEHMDA quantitatively.

SA-t-PEHMDA (6 mmol) was added to a 250 ml rbf followed by addition of cis-norbornene-exo-2,3-dicarboxylic anhydride (6.5 mmol), toluene (120 ml) and triethylamine (6 mmol). The mixture was then refluxed overnight with a dean stark trap connected, for water removal. The mixture was then cooled and concentrated, followed by addition of MeOH, to give a beige precipitate. The mixture was filtered and residue was washed with MeOH before drying in a vacuum oven overnight, to give SA-t-PE macromonomer (SA-t-PEHMDANB) quantitatively. ¹H NMR (C₇D8) 5=5.88-5.85 (m, 2H), 5.16-5.47 (m, 2H), 3.41-3.36 (m, 5H), 2.97-3.36 (m, 4H), 2.64 (bs, 2H).

### 7.6.3. Synthesis of NBPEG Macromonomer Body (for H₂N-PEG-NH₂1,000-6,000)

PEG diamine (1 g) and cis-norbornene-exo-2,3-dicarboxylic anhydride (1 eq.) were added to a 100 ml rbf, followed by toluene (50 ml). Triethylamine (1 eq.) was added and the mixture stirred under reflux overnight, with a dean stark trap attached for water removal. The resulting solution was evaporated to dryness and dichloromethane (40 ml) was added, followed by 0.1 M HCl (40 ml). The organic layer was extracted and washed with 0.1 M NaOH (50 ml). 0.1 M NaOH (50 ml) was added to the aqueous fraction from the acid wash followed by CH₂Cl₂ (30 ml). The organic layer was extracted and combined, washed with sat. NaCl before drying over Na₂SO₄. The material was evaporated to dryness to give a pale orange oil, NBPEG for PEG diamine 1,000 and beige solid for PEG diamine 3,400 and 6,000. ¹H NMR (MeOD): δ=6.36 (t, 2H, NB), 3.67 (s, PEG), 3.21 (s, 2H, NB), 2.74 (s, 2H, NB), 1.92 (s, 2H).

### 7.6.4. Synthesis of NBPEG₁₀₀₀RGD as Representative Preparation for PEG 1,000-6,000

RGD (with 1 carboxylic acid on aspartic acid protected with OMe) (0.0937 g, 0.26 mmol), was dissolved in MeOH (2.5 ml) in a 4 ml vial, in the glovebox. ⁱPr₂EtN (91 μL, 0.52 mmol) was added and the mixture stirred (A). HOBT (0.0353 g, 0.26 mmol) and HBTU (0.0992 g, 0.26 mmol) were dissolved in MeOH (12.5 ml) in a 20 ml vial at 40° C., followed by addition of the RGD solution from (A), to give solution (B). Solution B is then added to NBPEG₁₀₀₀ (0.25 g, 0.218 mmol) in a 40 ml vial and stirred at rt overnight. The resultant mixture was then evaporated to dryness and the oil was added to diethylether (50 ml). The diethylether solution was chilled in a freezer for 48 h and decanted. MeOH (5 ml) was added to the residue to give an orange solution with white ppt. The mixture was passed through a syringe filter and the clear filtrate was evaporated to dryness to give an orange oil of RGDPEGNB at 95% yield. ¹H NMR (MeOD): δ=7.74 (dd), 7.35-7.42 (m), 6.32 (t), 4.39 (s), 4.20 (s), 3.63 (br, s), 3.60 (d), 3.17 (t), 2.70 (d). MALDI-MS: 661.3 ([M−NB]+2H⁺).

### 7.6.5. Synthesis of NBPEG₁₀₀₀DGEA as Representative Preparation for PEG 1,000-6,000

DGEA (with 2 carboxylic acid end protected with OMe) (0.109 g, 0.26 mmol) was dissolved in MeOH (2.5 ml) in glovebox. ⁱPr₂EtN (91 μl, 0.52 mmol) was added and mixture stirred as solution A. HOBT (0.0353 g, 0.26 mmol) and HBTU (0.0992 g, 0.26 mmol) were dissolved in MeOH (12.5 ml) at 40° C., followed by addition of the solution A to give suspension B. Suspension B was then added to NBPEGNH₂ (0.25 g, 0.218 mmol) and stirred at r.t. for 24 h. The resulting pale yellow mixture was then concentrated by solvent evaporation to give yellow oily mixture. The mixture was dispersed into Et₂O and the solution placed in a freezer for 48 h. The Et₂O layer was withdrawn and MeOH was added to the residue to give a yellow suspension. Yellow oily product NB-PEG-DGEA (0.28 g, yield 73%) was obtained upon filtration and solvent evaporation. ¹H NMR (CD₃OD, 500 MHz, 25° C.): δ 7.80 (d, 1H), 7.71 (d, 1H), 7.44-7.38 (m, 2H), 6.33 (s, 2H), 4.40 (s, 2H), 4.22 (s, 1H), 3.95 (s, 1H), 3.68 (m, 6H), 3.64 (m, 84H), 3.57 (m, 4H), 3.18 (s, 2H), 2.82 (s, 2H), 2.72 (s, 2H), 2.47 (m, 2H), 2.14 (m, 1H), 1.96 (m, 1H), 1.48-1.41 (dd, 2H).

### 7.6.6. Synthesis of NBPEG₁₀₀₀(GPHyp)₃ as Representative Preparation for Collagen Fragments of Glycine, Proline and Hydroxyproline in Varying Sequence and Chain Length Up to n=6, PEG 1,000-6,000

(GPHyp)₃ (0.213 g, 0.26 mmol) was dissolved in MeOH (2.5 ml) in glovebox. ⁱPr₂EtN (91 μl, 0.52 mmol) was added and mixture stirred (solution A). HOBT (0.0353 g, 0.26 mmol) and HBTU (0.0992 g, 0.26 mmol) were dissolved in MeOH (12.5 ml) at 40° C., followed by addition of the solution A to give suspension B. Suspension B was then added to NBPEGNH₂ (0.25 g, 0.218 mmol) and stirred at r.t. for 24 h. The resulting pale yellow mixture was then concentrated by solvent evaporation to give beige mixture. The mixture was dispersed into Et₂O and freezed for 48 h. The Et₂O layer was withdrawn and MeOH was added to the residue to give a beige suspension. Beige oily product NB-PEG-(GPHyp)₃ (0.25 g, yield 50%) was obtained upon filtration and solvent evaporation.

¹H NMR (CD₃OD, 500 MHz, 25° C.): δ 6.33 (s, 2H), 4.73-4.44 (br, 4H), 3.65 (m, 84H), 3.57 (m, 4H), 3.18 (s, 2H), 2.72 (s, 2H), 2.39-1.80 (br, 8H), 1.44-1.37 (dd, 2H).

### 7.6.7. Synthesis of NBPEG₁₀₀₀(IRIK)₂ as Representative Preparation for PEG 1,000-6,000

(IRIK)₂ (with 1 Boc-protected amine) (0.100 g, 0.0875 mmol) was dissolved in MeOH (1.0 ml) in glovebox. ⁱPr₂EtN (31 μl, 0.175 mmol) was added and mixture stirred as solution A. HOBT (0.012 g, 0.0875 mmol) and HBTU (0.033 g, 0.0875 mmol) were dissolved in MeOH (1.5 ml) at 40° C., followed by addition of the solution A to give suspension B. Suspension B was then added to NBPEGNH₂ (0.0835 g, 0.073 mmol) and stirred at r.t. for 24 h. The resulting pale yellow mixture was then concentrated by solvent evaporation to give yellow oily mixture. The mixture was dispersed into Et₂O and the solution placed in a freezer for 48 h. The Et₂O layer was withdrawn and MeOH was added to the residue to give a yellow suspension. Yellow sticky solid product (0.14 g, yield 70%) was obtained upon filtration and solvent evaporation. The solid was dissolved in dichloromethane (3.0 ml) and trifluoroacetic acid (0.5 ml) and stirred at r.t. for 24 h. The resulting pale yellow mixture was then concentrated by solvent evaporation and washed with Et₂O to give pale yellow solid. The solid was re-dissolved in dichloromethane (2.0 ml) and MeOH (1.0 ml). Et₃N (200 μl) was added and the mixture stirred at r.t. for 24 h. The resulting pale yellow mixture was then concentrated by solvent evaporation and washed repeatedly with Et₂O to give pale yellow solid NB-PEG-IRIK (0.12 g, 63%). ¹H NMR (CD₃OD, 500 MHz, 25° C.): δ 6.32 (s, 2H), 4.50-4.10 (m, 7H), 3.64 (m, 84H), 3.25-3.10 (m, 4H), 3.00-2.80 (m, 5H), 1.90-1.30 (m, 30H), 1.25-1.15 (m, 4H), 1.05-0.85 (m, 24H).

### 7.6.8. Procedure for ROMP of SA-t-PE Macromonomer and RGD Peptide Macromonomer as Representative Procedure for SA-t-PE-Peptide Copolymers, for Peptide Length of 3-20 Amino Acids in any Sequence

RGD peptide macromonomer (0.0342 g, 0.0023 mmol) was weighed into a 20 ml vial followed by addition of SA-t-PE (0.2 g, 0.12 mmol). Benzene (2.3 ml) was added and the mixture stirred at 75° C. till a clear solution was obtained. A solution of catalyst 1 in benzene (1.25 mol %, 0.05 M) was added to the solution and the reaction was stirred for 22 h at 75° C. Ethyl vinyl ether was added to the reaction mixture followed by MeOH (15 ml) to give a beige precipitate. The mixture is filtered and the residue washed 5 times with MeOH before being dried overnight in a vacuum oven.

For SA-t-PERGD copolymer, ¹H NMR: 6.0 (s, unreacted SA-t-PE), 5.62-5.38 (m), 3.65 (PEG), 1.46 (t, CH₂), 1.02 (PE CH₃).

### Example 8: Amine-Terminated Polyethylene (PE)-Peptide Copolymers

### 8.1. Amine-Terminated PE-RGD Copolymer

This example shows a one-pot, 2-step strategy to create a primary amine terminus on polyethylene and its subsequent use in macromonomer synthesis, followed by copolymerization with a biomacromonomer to form bioactive polyethylene copolymers. In this method, linear primary amines were created using polyethylene via hydroaminomethylation and subsequently used in macromonomer formation with cis-norbornene-exo-2,3-dicarboxylic anhydride. Bioactive polyethylene can be created by copolymerizing this PE macromonomer with biomolecule-containing macromonomers. PE-RGD copolymers are reported here as an example of a bioactive PE that is compatible with human skin fibroblasts.

### 8.1.1. PE Macromonomer

To obtain a primary amine terminus on polyethylene (PE) for macromonomer formation, hydroaminomethylation of vinyl terminated PE was carried out in a one pot, two-step process where it was first converted into a linear carbonyl via hydroformylation, followed by reductive amination in the presence of hexamethylenediamine (HMDA) or NH₃ gas (Scheme 4.2). The linear to branch ratio here is excellent where branched carbonyl or amine was not detected. Negligible amounts of linear alcohol (<1%) was detected in the crude product.

Upon obtainment of the amine terminated PE, condensation with cis-norbornene-exo-2,3-dicarboxylic anhydride, afforded the PE macromonomer (Scheme 4.3), which could be used in brush polymer formation with either itself or other macromonomers.

### 8.1.2. PE-Peptide Copolymer

To demonstrate formation of bioactive PE using the PE macromonomer, PE macromonomer was reacted with a pegylated RGD macromonomer to obtain a copolymer of PE and pegylated RGD, using ring opening metathesis polymerization (ROMP) with a Grubbs' catalyst (Scheme 8.1).

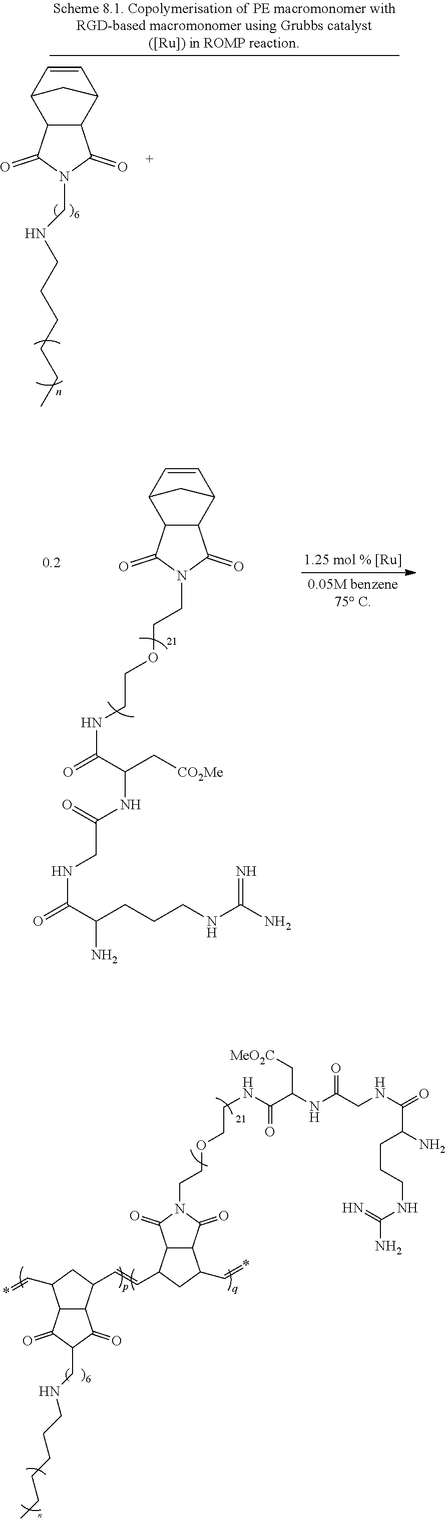

### 8.2. Thermal Stability

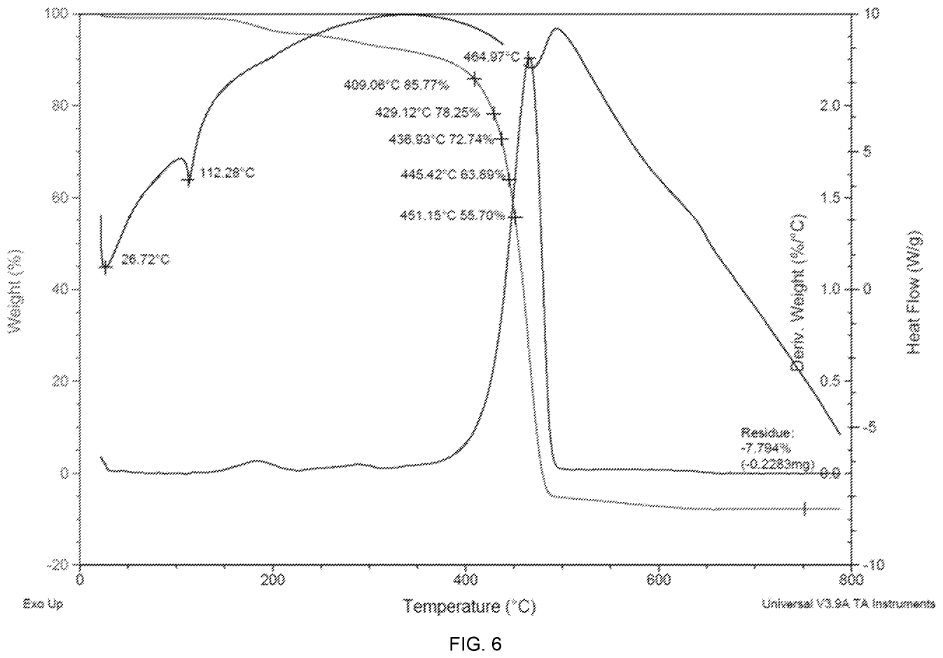

The PERGD copolymer was checked for thermal stability before undergoing material processing. From TGA analysis of the copolymer, it can be seen that the polymer undergoes 50% weight loss at 465° C., indicating its high thermal stability compared to pure RGD itself which has a degradation temperature of less than 200° C. (FIG. 6).

### 8.3. Biocompatibility

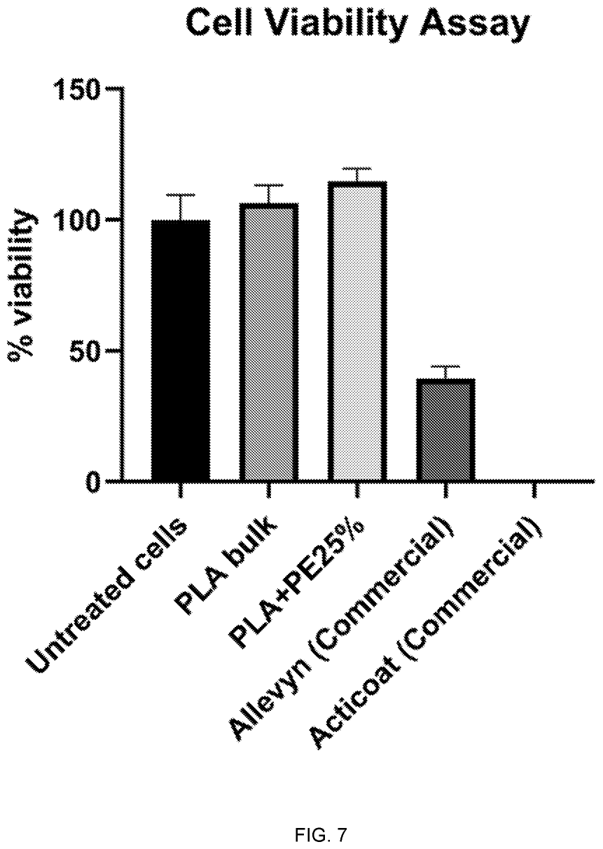

Subsequently, PERGD copolymer was blended with medical grade PLA and electrospun into nanofibers for biocompatibility tests with Hs27 human fibroblasts grown in Dulbecco's Modified Eagle Medium (DMEM) w/10% FBS and 1% Pen/Strep. From the 72 h cell viability tests on electrospun samples, it can be seen that PERGD shows slight cell proliferation over negative control or pure PLA at 25% PERGD/PLA blending ratio and much better cell viability than commercial wound dressings Acticoat and Allevyn (FIG. 7). Viable cells are essential for cell proliferation.

In conclusion, amine terminated PE was successfully created using a one pot, 2-step hydroaminomethylation reaction in the presence of HMDA and PE macromonomers were constructed using it. Bioactive PE copolymer was also created and tested successfully for human skin biocompatibility, using PERGD as example.

### 8.4. Materials and Methods

### 8.4.1. General Procedure

Ring opening metathesis polymerization reactions and RGD macromonomer synthesis were carried out in a Vacuum Atmosphere glovebox under nitrogen atmosphere. PEHMDANB macromonomer was synthesized under ambient conditions. PEHMDA synthesis was carried out in a Hastelloy pressure reactor fitted with PTFE gasket, from Parr Instrument company. All the solvents used-anhydrous benzene and anhydrous methanol from Alfa Aesar, were used as purchased, in the glovebox. Grubbs second generation catalyst was purchased from Sigma Aldrich and all peptides were purchased from Biomatik Inc. [Rh(acac)(CO)₂], [Ir(COD)Cl]₂, xantphos, HMDA, triethylamine and PEG diamine were purchased from Alfa Aesar. All purchased reagents were used without further purification. Vinyl terminated PE (MW 1,400-5,000) was obtained according to the literature method (Macromolecules 2009, 42, 4356-4358; following the second example in Supporting Information).

¹H NMR spectra were recorded on a Bruker Avance 400 MHz NMR spectrometer. GPC chromatogram were recorded on an Agilent Infinity II High Temperature GPC system equipped with 2*PLgel Mixed B columns (300×7.5 mm, particle size 10 μm) and 1*PLgel Mixed B guard column (50×7.5 mm). Eluent is TCB with 1 ml/min flow rate and oven temperature of 160° C. Polystyrene was used as calibration standard.

The PERGD copolymers were blended with PLA at 1:4 ratio and electrospun into sheets of fibers, which were then sterilized with 70% ethanol, dried and incubated for 72 h with fibroblasts Hs27 before being checked for cell viability using Celltitre-Glo assays.

### 8.4.2. Synthesis of PEHMDA

Vinyl terminated PE (0.35 g, 0.25 mmol) and xantphos (0.0036 g, 6.25 μmol) were weighed into a 25 ml pressure reactor followed by addition of toluene (3.5 ml). Rh(acac)(CO)₂ was then added (0.5 ml, 0.65 mg/ml, 1.25 μmol) to the mixture. The vessel was sealed and flush 5 times with CO/H₂ (g) (1:1) and pressurized to 45 bar with the gas. The mixture was stirred at 100° C. for 12 h then cooled. HMDA (0.0581 g, 0.5 mmol) was dissolved in toluene (1 ml) before being added to the cooled mixture, followed by [Ir(COD)Cl]₂ (1 ml, 0.84 mg/ml). The vessel was sealed and flushed 5 times with H₂ (g) and pressurized to 20 bar with the gas. The mixture was stirred at 135° C. for 4h before being cooled, followed by addition of MeOH to result in a white precipitate. The precipitate was filtered and the residue washed repeatedly with MeOH, followed by drying in a vacuum oven to yield a white solid product of PEHMDA at 68% yield. ¹H NMR (toluene-d8, 90° C.): δ=0.91 (t, 3H), 1.36 (br, s, 148H), 1.47-1.44 (m), 2.61-2.54 (m, 6H at 68% conversion).

### 8.4.3. Synthesis of PECH₂NH₂

Vinyl terminated PE (0.35 g, 0.25 mmol) and xantphos (0.0036 g, 6.25 μmol) were weighed into a 50 ml pressure reactor followed by addition of toluene (3.5 ml). Rh(acac)(CO)₂ was then added (0.5 ml, 0.65 mg/ml, 1.25 μmol) to the mixture. The vessel was sealed and flush 5 times with CO/H₂ (g) (1:1) and pressurized to 45 bar with the gas. The mixture was stirred at 100° C. for 12 h then cooled. [Ir(COD)Cl]₂ (1 ml, 0.84 mg/ml) was added to the cooled mixture and the vessel was sealed, flushed with NH₃ (g) 5 times before being pressurized to 3 bar with NH₃ (g). The vessel was further pressurized with another 20 bar H₂ (g). The mixture was then stirred at 135° C. for 3 h before being cooled, followed by addition of MeOH to result in a white precipitate. The precipitate was filtered and the residue washed repeatedly with MeOH, followed by drying in a vacuum oven to yield a white solid product of PECH₂NH₂ at 61% yield. ¹H NMR (toluene-d8, 90° C.): δ=0.90 (t, 3H), 1.34 (br, s, 194H), 2.58 (t, 2H, at 61% yield).

### 8.4.4. Synthesis of PEHMDANB

PEHMDA and PECH₂NH₂ cannot be separated from unreacted PE and is used as a mixture. Quantities of PEHMDA and PECH₂NH₂ used are calculated based on percentage of each in sample mixture containing unreacted PE.

PEHMDA (0.2 mmol) and cis-norbornene-exo-2,3-dicarboxylic anhydride (0.04 g, 0.25 mmol) were weighed into an rbf, followed by addition of toluene (20 ml) and Et₃N (28 μL, 0.2 mmol). The flask was equipped with a dean stark trap and the mixture refluxed for 12 h. The reaction was cooled and MeOH was added to the mixture to give a white precipitate in a pale yellow solution. The mixture was filtered and the residue washed with MeOH repeatedly to yield an off white product. ¹H NMR (toluene-d8, 90° C.): 5.89 (s), 5.87 (s), 3.42 (t), 3.01 (t), 2.53-2.60 (m), 2.19 (s), 1.37 (br s), 0.92 (t). Product exists a mixture of PEHMDANB and unreacted PE.

### 8.4.5. Procedure for ROMP of PEHMDANB Macromonomer and RGD Peptide Macromonomer as Representative Procedure for PE-Peptide Copolymers, for Peptide Length of 3-20 Amino Acids in any Sequence

RGD peptide macromonomer (0.0342 g, 0.0023 mmol) is weighed into a 20 ml vial followed by addition of PEHMDANB (0.12 mmol). Benzene (2.3 ml) is added and the mixture stirred at 75° C. till a clear solution is obtained. A solution of Grubbs' catalyst (2^{nd} generation) in benzene (1.25 mol %, 0.05 M) is added to the solution and the reaction is stirred for 22 h at 75° C. Ethyl vinyl ether is added to the reaction mixture followed by MeOH (15 ml) to give a beige precipitate. The mixture is filtered and the residue washed 5 times with MeOH before being dried overnight in a vacuum oven. For PERGD, ¹H NMR (toluene d8, 90° C.): δ=5.88 (s), 5.85 (s) (unreacted PEHMDANB), 5.46-5.42 (m), 3.53 (br s, PEG), 3.4 (t), 2.57-2.53 (m), 1.36 (br s, PE CH₂), 0.91 (br s, PE CH₃). Sample contains unreacted PE.

### Example 9: Applications

The present disclosure provides a new modular synthesis method to create softer and more biocompatible PE/PP blends. Embodiments of the bioactive polyethylene copolymer disclosed herein possess one or more of the following properties:
non-toxic to skin;
increased thermal stability for material processing compared to pure collagen which denatures at 37° C.; and
increased structure integrity compared to pure collagen, oligopeptides or oligosaccharides, which exists in gels or hygroscopic crystals.

Advantageously, embodiments of the bioactive polyethylene copolymer disclosed herein allow for biomolecule (e.g., collagen) to be blended into base material of synthetic polymer similar to the synthetic polymer side arms of copolymer (e.g., polypropylene) without phase separation, despite the opposing material properties existing between the hydrophobic PP and hydrophilic collagen.

Embodiments of the bioactive polyethylene copolymer disclosed herein showed good thermal stability and biocompatibility data. Advantageously, embodiments of the bioactive polyethylene copolymer disclosed herein can be used to make consumer care products such as diapers and sanitary products or biomedical devices such as joint implants, gut stents, wound dressings, cartilage implants.

The present disclosure provides a new modular synthesis method to create bioactive macromonomers rapidly for construction of bioactive copolymers with bioactive molecule of choice, depending on the targeted application or bioactivity required. Bioactive macromonomers may be easily copolymerized with polyethylene to form bioactive polyethylene copolymers with desired physical and mechanical properties. Advantageously, there is increased stability of bioactive molecule upon connection to polymer linker. Embodiments of the strategy disclosed herein allow for any peptide, carbohydrate or drug molecule to be used in polymer synthesis without loss of bioactivity. Embodiments of the strategy disclosed herein also allow rapid build up of bioactive macromonomer library. Any bioactive molecule with a carboxylic acid group may be used. In summary, the present disclosure provides a highly versatile strategy for biomedical material customization.

Embodiments of the method disclosed herein allow macromonomers to be paired with synthetic polymer of choice to create bioactive polymer that has both mechanical and physical properties of synthetic polymer and biological activity of bioactive molecule.

Embodiments of the method disclosed herein is an easy strategy to create different types of bioactive polymers that are chemically bonded instead of physical blends of bioactive molecules into synthetic polymers.

Advantageously, non-cell or growth factor-based bioactivity is/are provided on the polymer disclosed herein. Embodiments of the bioactive polyethylene copolymer disclosed herein possess both bioactivity to enhance therapeutic effects such as tissue regeneration, biofilm eradication etc, and also structural integrity and mechanical strength, like a polymer. Embodiments of the bioactive polyethylene copolymer disclosed herein allow for biomolecule to be blended into base material of synthetic polymer similar to the synthetic polymer side arms of copolymer, without phase separation. Embodiments of the method disclosed herein allow the polyethylene to become biocompatible to human tissues upon modification with biomolecules. Embodiments of the method disclosed herein allow a wide range of biomolecules to be used to achieve any desired therapeutic effect. Embodiments of the method disclosed herein also allow a good range of synthetic polymers to be used to achieve different mechanical, physical properties required in material for targeted biodevice.

Embodiments of the bioactive polyethylene copolymers disclosed herein may be used as bioadditives for biomedical devices to provide therapeutic effects to device material itself.

Embodiments of the method disclosed herein use non cell- or growth factor-based therapy, which allow for long shelf life of device or materials such as scaffold and prevent unwanted or uncontrolled bioactivity (for e.g., tissue regeneration).

Embodiments of the bioactive polyethylene copolymers disclosed herein may be used as bioadditives for wound dressings, cartilage implants or bone scaffold to create stimulus required for skin, cartilage or bone tissue regeneration.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A bioactive polyethylene copolymer with a poly(norbornene) backbone comprising one or more repeating units represented by general formula (I) and one or more repeating units represented by general formula (II): 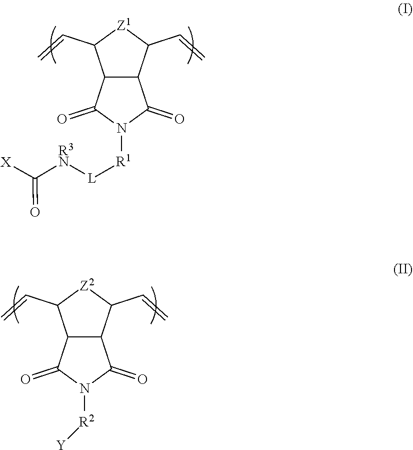
wherein
R¹ is optionally substituted alkyl;
R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;
R³ is selected from H, optionally substituted alkyl, optionally substituted alkenyl or optionally substituted alkynyl;
L is heteroalkylene;
X comprises a bioactive moiety selected from the group consisting of proteins, peptides, carbohydrates, therapeutic/drug molecules and derivatives thereof,
Y comprises polyethylene or parts thereof, and
Z¹ and Z² are each independently selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl.

2. The copolymer of claim 1, wherein Y is represented by general formula (III): 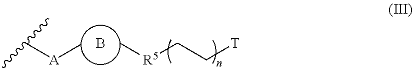
wherein
A is optionally present as NR^{c}, wherein R^{c} is independently selected from H, optionally substituted alkyl, optionally substituted alkenyl or optionally substituted alkynyl;
B is optionally present as a 5-membered or 6-membered heterocyclic ring having at least one N heteroatom in the ring;
R⁵ is selected from an optionally substituted alkyl, optionally substituted alkenyl, optionally substituted alkynyl, optionally substituted alkoxyalkyl, optionally substituted alkylcarbonyl or optionally substituted alkylcarbonylalkyl;
T is a terminal group selected from the group consisting of hydrogen and methyl; and
n is from 10 to 350.

3. The copolymer of claim 2, wherein n is from 20 to 250.

4. The copolymer of claim 2, wherein B is present and represented by the following structure: 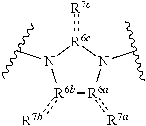
wherein
R^{6a}, R^{6b}, R^{6c} and R^{6d} are each independently selected from the group consisting of C, CR^{a}, CR^{a}R^{b}, N, NR^{c}, O or S, wherein R^{a}, R^{b}, and R^{c} are each independently selected from the group consisting of H, optionally substituted alkyl, optionally substituted alkenyl and optionally substituted alkynyl; and
R^{7a}, R^{7b} and R^{7c} are optionally present as ═O, ═S, —F, —Cl, —Br, —I, ═CR^{a}R^{b}, —CR^{a}R^{b}R^{c}, —OH, —SH, —NH₂ or ═NR^{c}.

5. The copolymer of claim 1, wherein Y is selected from the following general formulae (IIIa), (IIIb) or (IIIc): 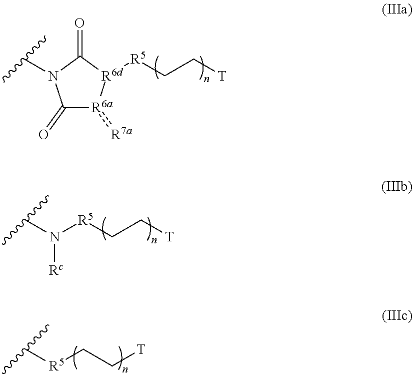
wherein
R⁵ is selected from the group consisting of C₁-C₂₀ alkyl, C₂-C₂₀ alkenyl, C₂-C₂₀ alkynyl, C₁-C₂₀ alkoxy, C₁-C₂₀ alkoxyalkyl, C₂-C₂₀ alkylcarbonyl and C₃-C₂₀ alkylcarbonylalkyl;
R^{6a} and R^{6d} are each independently selected from the group consisting of C, CR^{a}, CR^{a}R^{b}, N, NR^{c}, O or S, wherein R^{a}, R^{b}, and R^{c} are each independently selected from the group consisting of H, optionally substituted alkyl, optionally substituted alkenyl and optionally substituted alkynyl;
R^{7a} is optionally present as ═O, ═S, —F, —Cl, —Br, —I, ═CR^{a}R^{b}, —CR^{a}R^{b}R^{c}, —OH, —SH, —NH₂ or ═NR^{c};
T is a terminal group selected from the group consisting of hydrogen and methyl; and
n is from 10 to 350.

6. The copolymer of claim 1, wherein Y is selected from the following general formulae (IIId), (IIIe) or (IIIf): 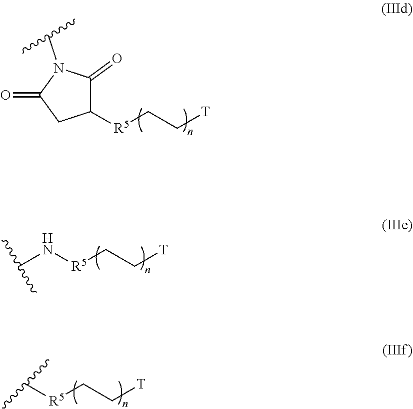
wherein
R⁵ is selected from the group consisting of C₁-C₂₀ alkyl, C₂-C₂₀ alkenyl, C₂-C₂₀ alkynyl, C₁-C₂₀ alkoxy, C₁-C₂₀ alkoxyalkyl, C₂-C₂₀ alkylcarbonyl and C₃-C₂₀ alkylcarbonylalkyl;
T is a terminal group selected from the group consisting of hydrogen and methyl; and
n is from 10 to 350.

7. The copolymer of claim 1, wherein the repeating unit represented by general formula (I) is in an amount of from 1 to 100 molar % relative to the copolymer.

8. The copolymer of claim 1, wherein the molecular weight of general formula (I) do not differ from the molecular weight of general formula (II) by more than 30% of the molecular weight of general formula (II).

9. The copolymer of claim 1, wherein L is heteroalkylene having from 20 carbon atoms to 300 carbon atoms.

10. The copolymer of claim 1, wherein L is polyethylene glycol (PEG), optionally wherein L is polyethylene glycol (PEG) having a number average molecular weight of between 500 and 7,000.

11. (canceled)

12. The copolymer of claim 1, wherein R¹ is C₁-C₄ alkyl and R² is selected from C₁-C₂₀ alkyl, C₂-C₂₀ alkenyl, C₂-C₂₀ alkynyl, C₁-C₂₀ alkoxy, C₁-C₂₀ alkoxyalkyl, C₂-C₂₀ alkylcarbonyl or C₃-C₂₀ alkylcarbonylalkyl, optionally wherein R¹ is straight or branched C₁-C₄ alkyl substituents independently selected from methyl, ethyl, n-propyl, 2-propyl, isopropyl, n-butyl, isobutyl, sec-butyl or t-butyl, and R² is straight or branched C₁-C₂₀ alkyl substituents independently selected from methyl, ethyl, n-propyl, 2-propyl, isopropyl, n-butyl, isobutyl, sec-butyl, t-butyl, hexyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl or decyl.

13. (canceled)

14. The copolymer of claim 1, wherein Z¹ and Z² are both CR^{a}R^{b} wherein R^{a} and R^{b} are each independently selected from the group consisting of H, optionally substituted alkyl, optionally substituted alkenyl and optionally substituted alkynyl.

15. The copolymer of claim 1, wherein X comprises protein, peptide or carbohydrate selected from the group consisting of peptide sequence, laminin-derived peptide, integrin binding peptide, cell-penetrating peptide, collagen sequence, collagen mimics, collagen fragment, heparin sulfate, glycosaminoglycans (GAGs) and derivatives thereof.

16. The copolymer of claim 1, wherein X is selected from the group consisting of RGD, SRGDS (SEQ ID NO: 1), RGDS (SEQ ID NO: 2), A5G81 (AGQWHRVSVRWGC) (SEQ ID NO: 3), SVVYGLR (SEQ ID NO: 4), (IRIK)₂ (SEQ ID NO: 6), (IKKI)₃ (SEQ ID NO: 7), DGEA (SEQ ID NO: 5), (PHypG)ₙtype sequence, (PGHyp)ₙ type sequence, (HypGP)ₙ type sequence, (HypPG)ₙ type sequence, (GHypP)ₙ type sequence, (GPHyp)ₙ type sequence, heparin oligosaccharide DP8, DP10, DP12, DP14, DP16 and hyaluronic acid.

17. A method of preparing a bioactive polyethylene copolymer of claim 1, the method comprising:
polymerising one or more bioactive macromolecules represented by general formula (IV) with one or more polyethylene macromolecules represented by general formula (V) in the presence of a catalyst to obtain the bioactive polyethylene copolymer: 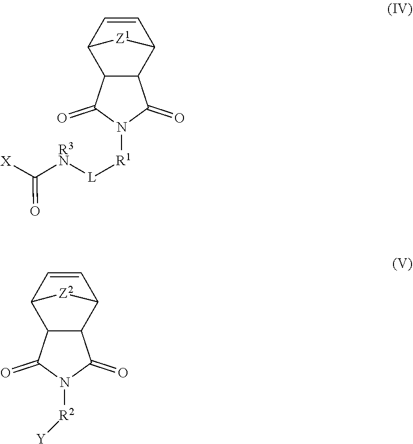
wherein
R¹ is optionally substituted alkyl;
R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;
R³ is selected from H, optionally substituted alkyl, optionally substituted alkenyl or optionally substituted alkynyl;
L is heteroalkylene;
X comprises a bioactive moiety selected from the group consisting of proteins, peptides, carbohydrates, therapeutic/drug molecules and derivatives thereof,
Y comprises polyethylene or parts thereof, and
Z¹ and Z² are each independently selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl.

18. The method according to claim 17, wherein the catalyst comprises a ruthenium complex.

19. The method according to claim 17, wherein the method comprises ring opening metathesis polymerisation (ROMP).

20. (canceled)

21. The method according to claim 17, wherein the method further comprises, prior to polymerising, preparing a polyethylene macromolecule represented by general formula (VIII) by: 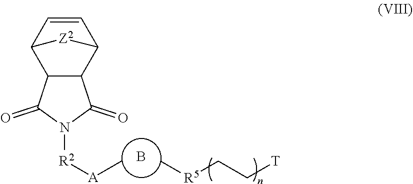
(i) providing a dicarboxylic anhydride having general formula (IX): 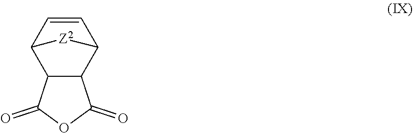
wherein Z² is selected from CR^{a}R^{b}, O, NR^{c}, SiR^{a}R^{b}, PR^{a} or S, wherein R^{a}, R^{b}, and R^{c} are each independently selected from the group consisting of H, optionally substituted alkyl, optionally substituted alkenyl and optionally substituted alkynyl; and
(ii) reacting said dicarboxylic anhydride having general formula (IX) with an amine to obtain the polyethylene macromolecule, the amine is represented by general formula (X): 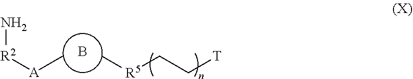
wherein
R² is selected from a single bond, optionally substituted alkyl, optionally substituted alkenyl, optionally substituted alkynyl, optionally substituted alkoxyalkyl, optionally substituted alkylcarbonyl or optionally substituted alkylcarbonylalkyl;
A is optionally present as NR^{c}, wherein R^{c} is independently selected from H, optionally substituted alkyl, optionally substituted alkenyl or optionally substituted alkynyl;
B is optionally present as a 5-membered or 6-membered heterocyclic ring having at least one N heteroatom in the ring;
R⁵ is selected from the group consisting of C₁-C₂₀ alkyl, C₂-C₂₀ alkenyl, C₂-C₂₀ alkynyl, C₁-C₂₀ alkoxy, C₁-C₂₀ alkoxyalkyl, C₂-C₂₀ alkylcarbonyl and C₃-C₂₀ alkylcarbonylalkyl;
T is a terminal group selected from the group consisting of hydrogen and methyl;
n is from 10 to 350,
optionally wherein the method further comprises, prior to (ii),
(a-i) providing a polyethylene having general formula (XIa) or (XIb): 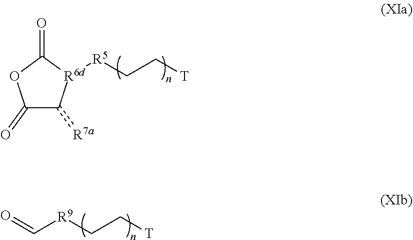
wherein
R^{6a} and R^{6d} are each independently selected from the group consisting of C, CR^{a}, CR^{a}R^{b}, N, NR^{c}, O or S, wherein R^{a}, R^{b}, and R^{c} are each independently selected from the group consisting of H, optionally substituted alkyl, optionally substituted alkenyl and optionally substituted alkynyl;
R^{7a} is optionally present as ═O, ═S, —F, —Cl, —Br, —I, ═CR^{a}R^{b}, —CR^{a}R^{b}R^{c}, —OH, —SH, —NH₂ or ═NR^{c};
R⁸ and R⁹ are each independently selected from the group consisting of C₁-C₂₀ alkyl, C₂-C₂₀ alkenyl, C₂-C₂₀ alkynl, C₁-C₂₀ alkoxy, C₁-C₂₀ alkoxyalkyl, C₂-C₂₀ alkylcarbonyl and C₃-C₂₀ alkylcarbonylalkyl;
T is a terminal group selected from the group consisting of hydrogen and methyl;
n is from 10 to 350; and
(b-i) reacting said polyethylene having general formula (XIa) or (XIb) with a diamine H₂N—R²—NH₂ or ammonia NH₃ to obtain the amine having general formula (X),
optionally wherein at least one of (ii) and (b-i) is performed in the presence of an organic solvent and/or a base, and
optionally wherein the organic solvent comprises an aromatic solvent; and the base comprises a tertiary amine.

22.-24. (canceled)

25. A material comprising a copolymer of claim 1 for use in medicine.

26. The material according to claim 25, wherein the material is part of an apparatus selected from the group consisting of consumer care products, wound dressing, skin scaffold, bone and bone marrow organoid scaffold, cartilage implant, joint implant and medical device.
